(19) United States
(12) Patent Application Publication
(10) Pub. No.: US 2019/0194684 A1
(43) Pub. Date: Jun. 27, 2019

(54) PARTHENOCARPY REGULATION GENE AND USE THEREOF

(71) Applicant: INCORPORATED ADMINISTRATIVE AGENCY NATL. AGRICULTURE AND FOOD RESEARCH ORG., Ibaraki (JP)

(72) Inventors: Tsukasa Nunome, Tsu-shi (JP); Ichiro Honda, Maebashi-shi (JP); Akio Ohyama, Tsu-shi (JP); Hiroyuki Fukuoka, Tsu-shi (JP); Hirotaka Yamaguchi, Tsu-shi (JP); Koji Miyatake, Tsu-shi (JP)

(73) Assignee: INCORPORATED ADMINSTRATIVE AGENCY NATL. AGRICULTURE AND FOOD RESEARCH ORG., Ibaraki (JP)

(21) Appl. No.: 14/418,504

(22) PCT Filed: Jul. 31, 2013
(86) PCT No.: PCT/JP2013/070801

§ 371 Date: Mar. 27, 2015

(30) Foreign Application Priority Data
Jul. 31, 2012 (JP) 2012-170506

(51) Int. Cl. C12N 15/82; C07K 14/415; G01N 33/50

(52) U.S. Cl. CPC C12N 15/8287; G01N 33/5097; C07K 14/415

(57) ABSTRACT

The present invention provides a parthenocarpy regulatory gene including any of the following polynucleotides and use of the parthenocarpy regulatory gene. (1) A polynucleotide that encodes an amino acid sequence represented by SEQ ID NO: 1, (2) a polynucleotide that encodes an amino acid sequence having a sequence identity of 70% or higher relative to the amino acid sequence represented by SEQ ID NO: 1, (3) a polynucleotide that encodes an amino acid sequence in which 1 to 87 amino acids are substituted and/or the like in the amino acid sequence represented by SEQ ID NO: 1, and (4) a polynucleotide that is hybridized with a polynucleotide having a sequence complementary to the polynucleotide described in the above (1) under a stringent condition.

### TECHNICAL FIELD

The present invention relates to a parthenocarpy regulatory gene and a method for producing a plant with the use of the parthenocarpy regulatory gene.

### BACKGROUND ART

Parthenocarpy is a trait that fruit is enlarged without pollination and/or fertilization. This trait is advantageous in terms of harvesting fruits without carrying out, for example, work such as pollination induction with the use of a flower visiting insect or fructification induction by plant chemical application.

Conventionally, with regard to tomato, a parthenocarpic tomato line has been distributed as a genetic resource, and breeding of a commercial cultivar has been carried out by the use of the parthenocarpic tomato line as a breeding material. Moreover, a pat-2 gene exists as a parthenocarpy-related gene of tomato, and the gene reportedly has a monogenic recessive trait.

Patent Literature 1 discloses a method for producing a seedless parthenocarpic tomato by crossbreeding (i) a tomato strain that includes at least one parthenocarpy-related gene as a male and (ii) a male sterile tomato strain that includes at least one parthenocarpy-related gene as a female. Further, Patent Literature 1 discloses the pat-2 as one of the parthenocarpy-related genes.

Patent Literature 2 discloses a method for creating a parthenocarpic tomato plant or a parthenocarpic and male sterile tomato plant, and the method includes introgressing of a genetic region from chromosome 4, 5 and/or 12 of S. habrochaites. Further, Patent Literature 2 discloses relation of the pat-2 to double recessive parthenocarpy.

### CITATION LIST

### Patent Literature

### [Patent Literature 1]

Japanese Patent Application Publication Tokukai No. 2011-045373 (Publication date: Mar. 10, 2011)

### [Patent Literature 2]

Japanese Translation of PCT International Application Tokuhyo No. 2010-527586 (Publication date: Aug. 19, 2010)

### SUMMARY OF INVENTION

### Technical Problem

As disclosed in Patent Literatures 1 and 2, in the broad sense of tomato, the existence of a gene that relates to parthenocarpy is genetically recognized. However, details of a chromosomal location and of a structure of such a gene have not been reported yet.

Depending on cultivation conditions or cultivar lines, phenotypic expression of parthenocarpy can be unstable, and therefore it is sometimes difficult to evaluate parthenocarpy if the evaluation is based only on appearance of a plant. Moreover, in order to evaluate parthenocarpy, much manpower and time are required for emasculation before anthesis, evaluation of fruit set, and the like.

When the gene responsible for parthenocarpy is homozygous, the parthenocarpy trait can be evaluated based on appearance of tomato because parthenocarpy is a recessive trait. Moreover, in order to obtain a F1 cultivar that has parthenocarpy trait, it is necessary to introduce parthenocarpy trait into both parents, and therefore breeding of tomato cultivar cannot be carried out efficiently.

The present invention is accomplished in view of the problems, and its object is (i) to identify a new gene that is responsible for parthenocarpy and (ii) to provide use of the new gene.

### Solution to Problem

The inventors of the present invention diligently studied for attaining the object and, as a result, the inventors of the present invention have succeeded to identify and isolate a new gene that is responsible for parthenocarpy, and the present invention has been thus accomplished.

That is, the present invention encompasses any of the following 1) through 6):

1) A method for evaluating parthenocarpy of a plant, the method including the step of: checking whether or not expression of a parthenocarpy regulatory gene, which includes the polynucleotide described in any of (1) through (4) below, is inhibited in the plant; or checking whether or not a function of the polypeptide, which is encoded by a parthenocarpy regulatory gene, is inhibited in the plant.
(1) A polynucleotide that encodes a polypeptide having an amino acid sequence represented by SEQ ID NO: 1, (2) a polynucleotide that encodes a polypeptide (i) having a sequence identity of 70% or higher relative to the amino acid sequence represented by SEQ ID NO: 1 and (ii) having a parthenocarpy regulation activity, (3) a polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and (4) a polynucleotide that (i) is hybridized with a polynucleotide, which has a sequence complementary to the polynucleotide described in the above (1), under a stringent condition and (ii) encodes a polypeptide having a parthenocarpy regulation activity.
2) A method for producing a parthenocarpy-regulated plant, the method including the step of: selecting a parthenocarpic plant by carrying out the method described in the above 1).
3) A method for producing a parthenocarpy-regulated plant, the method including the step of: inhibiting, in a plant, gene expression of a parthenocarpy regulatory gene that includes a polynucleotide described in any of (1) through (4) below; or inhibiting, in a plant, a function of a polypeptide encoded by a parthenocarpy regulatory gene.
(1) A polynucleotide that encodes a polypeptide having an amino acid sequence represented by SEQ ID NO: 1, (2) a polynucleotide that encodes a polypeptide (i) having a sequence identity of 70% or higher relative to the amino acid sequence represented by SEQ ID NO: 1 and (ii) having a parthenocarpy regulation activity, (3) a polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and (4) a polynucleotide that (i) is hybridized with a polynucleotide, which has a sequence complementary to the polynucleotide described in the above (1), under a stringent condition and (ii) encodes a polypeptide having a parthenocarpy regulation activity.
4) A parthenocarpy-regulated plant produced by the method described in the above 2) or 3).
5) A parthenocarpy regulatory gene that includes a polynucleotide described in any of (1) through (4) below.
(1) A polynucleotide that encodes a polypeptide having an amino acid sequence represented by SEQ ID NO: 1, (2) a polynucleotide that encodes a polypeptide (i) having a sequence identity of 70% or higher relative to the amino acid sequence represented by SEQ ID NO: 1 and (ii) having a parthenocarpy regulation activity, (3) a polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and (4) a polynucleotide that (i) is hybridized with a polynucleotide, which has a sequence complementary to the polynucleotide described in the above (1), under a stringent condition and (ii) encodes a polypeptide having a parthenocarpy regulation activity.
6) A polypeptide described in any of (1) through (4) below. (1) A polypeptide having an amino acid sequence represented by SEQ ID NO: 1, (2) a polypeptide (i) having a sequence identity of 70% or higher relative to the amino acid sequence represented by SEQ ID NO: 1 and (ii) having a parthenocarpy regulation activity, (3) a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and (4) a polypeptide that (i) is encoded by a polynucleotide which is hybridized, under a stringent condition, with a polynucleotide that has a sequence complementary to a polynucleotide for encoding the polypeptide described in the above (1) and (ii) has a parthenocarpy regulation activity.

### Advantageous Effects of Invention

It is possible to evaluate parthenocarpy trait of a plant or to produce a parthenocarpic plant by using, as an indicator or a target, a gene or a polypeptide encoded by the gene.

### BRIEF DESCRIPTION OF DRAWINGS

FIG. 1 is a view relating to an Example of the present invention and illustrating a linkage map of a region in which a parthenocarpy responsible gene A is located.

FIG. 2 is a schematic view illustrating a vector used to produce RNAi transgenic plant in an Example of the present invention.

FIG. 3 is a view relating to an Example of the present invention and illustrating, in regard to each of RNAi transgenic plants and non-transgenic plants, comparison between stigma-removed fruits and pollinated fruits in terms of mature fruit weight.

FIG. 4 is a view relating to an Example of the present invention and illustrating ratios of mature stigma-removed fruit weights of respective RNAi transgenic plants and non-transgenic plants, where a fruit weight of a pollinated fruit is assumed to 1.

FIG. 5 is a view relating to an Example of the present invention and illustrating a result of analyzing stage-specific and tissue-specific gene expressions of the parthenocarpy responsible gene A.

FIG. 6 is a view relating to an Example of the present invention and illustrating a structure of a cloning vector including a full length of the parthenocarpy responsible gene A.

FIG. 7 is a view relating to an Example of the present invention and illustrating a structure of a binary vector including a full length of the parthenocarpy responsible gene A.

FIG. 8 is a view relating to an Example of the present invention and illustrating a result of measuring mature fruit weights of stigma-removed fruits and pollinated fruits of transgenic tomatoes in each of which the parthenocarpy responsible gene A is expressed.

FIG. 9 is a view relating to an Example of the present invention and illustrating ratios of mature fruit weights of respective of stigma-removed fruits and pollinated fruits of the transgenic tomatoes in each of which the parthenocarpy responsible gene A is expressed, where a fruit weight of a pollinated fruit is assumed to 1.

FIG. 10 is a view relating to an Example of the present invention and illustrating a schematic configuration of an RNAi-induction vector of a parthenocarpy responsible gene SmA of eggplant.

FIG. 11 is a view relating to an Example of the present invention and illustrating a schematic configuration of an RNAi-induction binary vector of a parthenocarpy responsible gene SmA of eggplant.

FIG. 12 is a view relating to an Example of the present invention and illustrating a result of inhibiting expression of a parthenocarpy responsible gene SmA in eggplant by RNAi.

FIG. 13 is a view relating to an Example of the present invention and illustrating comparison in terms of expression pattern between the parthenocarpy responsible gene SmA of eggplant and the parthenocarpy responsible gene A of tomato.

FIG. 14 is a view relating to an Example of the present invention and illustrating a result of segregating traits of parthenocarpy and non-parthenocarpy in regard to tomato with the use of marker sequences.

### DESCRIPTION OF EMBODIMENTS

The following description will discuss an embodiment of the present invention. Note that the present invention is not limited to this.

[Definitions of Terms]

In this specification, “polynucleotide” can be reworded as “nucleic acid” or “nucleic acid molecule”, and is intended to be a polymer of nucleotides. Moreover, “base sequence” can be reworded as “nucleic acid sequence” or “nucleotide sequence”, and is intended to be a sequence of deoxyribonucleotide or a sequence of ribonucleotide, unless otherwise particularly noted.

In this specification, “polypeptide” can be reworded as “protein”.

In this specification, “parthenocarpy (parthenocarpic)” is intended to be a trait of a plant whose fruit is enlarged without pollination and/or fertilization.

In this specification, “heterozygote (heterozygous)” is intended to be a state in which different allelic genes are at corresponding gene loci on respective homologous chromosomes, and “homozygote (homozygous)” is intended to be a state in which identical allelic genes are at corresponding gene loci on respective homologous chromosomes.

In this specification, “tomato” is a concept that encompasses, in the broad sense, cultivated species “S. lycopersicum” and wild species “S. cheesmaniae, S. chilense, S. chmielewskii, S. galapagense, S. habrochaites, S. lycopersicoides, S. neorickii, S. pennellii, S. peruvianum, and S. pimpinellifolium”, and is intended to be “S. lycopersicum” in the narrow sense.

In this specification, “A and/or B” is a concept that includes both “A and B” and “A or B”, and can be reworded as “at least one of A and B”.

In this specification, a wording “polynucleotides are complementary to each other” is synonymous with “base sequences of respective polynucleotides are complementary to each other”.

[1. Parthenocarpy Regulatory Gene]

A parthenocarpy regulatory gene in accordance with the present invention encodes a polypeptide that has an activity (i.e., a parthenocarpy regulation activity) to regulate at least parthenocarpy. The polypeptide “that has an activity to regulate parthenocarpy” indicates that expression of a trait, i.e., parthenocarpy is inhibited by the presence of the polypeptide, that is, parthenocarpy is negatively regulated. Moreover, in a case where expression of the parthenocarpy regulatory gene of the present invention is inhibited or an activity of a polypeptide encoded by the parthenocarpy regulatory gene is inhibited, a plant shows a parthenocarpy trait or has a predisposition of the trait even though parthenocarpy is not shown.

The parthenocarpy regulatory gene of the present invention includes any of polynucleotides described in the following (1) through (4):

(1) A polynucleotide that encodes a polypeptide having an amino acid sequence represented by SEQ ID NO: 1.
(2) A polynucleotide that encodes a polypeptide (i) having a sequence identity of 70% or higher relative to the amino acid sequence represented by SEQ ID NO: 1 and (ii) having a parthenocarpy regulation activity. Note that the sequence identity of the amino acid sequence is preferably 75% or higher or 80% or higher, more preferably 85% or higher, more preferably 90% or higher, further preferably 95% or higher, particularly preferably 96% or higher, 97% or higher, 98% or higher, or 99% or higher. For example, a mutant gene derived from tomato and a homologous gene derived from a plant other than tomato are encompassed in this range.
(3) A polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity. Note that the number of amino acids which are substituted, deleted, inserted, and/or added is more preferably 1 to 72; or 1 to 58, more preferably 1 to 43, more preferably 1 to 29, further preferably 1 to 14; or 1 to 10, particularly preferably 1 to 5 or 6. Note that the deletion, substitution, and/or addition of amino acids can be, for example, (i) carried out by artificially introducing a mutation(s) by the use of site-directed mutagenesis such as a Kunkel method (Kunkel et al. (1985): Proc.Natl.Acad.Sci.USA, vol.82. p488-) or (ii) derived from a naturally occurring similar mutant polypeptide.

Moreover, a region in which amino acids are deleted, substituted, and/or added is preferably, in the amino acid sequence represented by SEQ ID NO: 1, a region except for (i) a part from 56th valine to 112th asparagine which is predicted as a Zn finger domain and (ii) a part from 219th lysine to 276th asparagine which is predicted as a homeo domain.

(4) A polynucleotide that (i) is hybridized with a polynucleotide, which has a sequence complementary to the polynucleotide described in the above (1), under a stringent condition and (ii) encodes a polypeptide having a parthenocarpy regulation activity. Note that the stringent condition is, for example, a condition described in Reference Literature [Molecular cloning-a Laboratory manual 2nd edition (Sambrook et al., 1989)]. Specifically, the stringent condition is, for example, a condition in which the polynucleotide is hybridized by being constantly heated, at 65° C. for 8 to 16 hours together with a probe, in a solution that contains 6×SSC (composition of 1×SSC: 0.15 M of sodium chloride, 0.015 M of sodium citrate, pH 7.0), 0.5% of SDS, 5×Denhardt's solution, and 100 mg/mL of herring sperm DNA. Note that the polynucleotide preferably has a sequence identity of 70% or higher, more preferably has a sequence identity of 75% or higher or 80% or higher, more preferably has a sequence identity of 85% or higher, further preferably has a sequence identity of 90% or higher, particularly preferably has a sequence identity of 95% or higher, 96% or higher, 97% or higher, 98% or higher, or 99% or higher, relative to the base sequence of the polynucleotide described in the above (1).

The parthenocarpy regulatory gene of the present invention can exist in a form of RNA (e.g., mRNA) or a form of DNA (e.g., cDNA or genomic DNA). The DNA can be double-stranded or single-stranded. The base sequence represented by SEQ ID NO: 2, which is an example of the polynucleotide of the present invention, is a structural gene part of cDNA that encodes the polypeptide represented by SEQ ID NO: 1. The parthenocarpy regulatory gene of the present invention can include an additional sequence such as a sequence of an untranslated region (UTR).

A method for obtaining (isolating) the parthenocarpy regulatory gene of the present invention is not limited to a particular one, and can be a method which includes, for example, (i) preparing a probe which is specifically hybridized with a part of the base sequence of the parthenocarpy regulatory gene and (ii) screening a genomic DNA library or a cDNA library.

Alternatively, the method for obtaining the parthenocarpy regulatory gene of the present invention can be a method which uses amplifying means such as PCR. For example, a large amount of DNA fragments each containing the parthenocarpy regulatory gene of the present invention can be obtained by (i) preparing primers from respective 5′ end and 3′ end sequences (or complementary sequences thereof) in cDNA of the parthenocarpy regulatory gene, (ii) carrying out PCR or the like with the use of the primers and genomic DNA (or cDNA) or the like as a template, and (iii) amplifying a DNA region between the primers.

An origin of the parthenocarpy regulatory gene of the present invention is not limited to a particular one, provided that the origin is a plant. Note, however, that the origin is preferably a solanaceous plant such as tomato, eggplant, pimiento, paprika, pepper, or potato, more preferably a solanum plant such as tomato, eggplant, or potato, further preferably tomato or eggplant, particularly preferably tomato.

Note that whether or not a candidate gene of the isolated parthenocarpy regulatory gene has an intended parthenocarpy regulation activity can be evaluated by observing whether or not parthenocarpy of the original plant is induced by inhibiting expression of the candidate gene in the original plant.

The parthenocarpy regulatory gene of the present invention can be used to elucidate a mechanism of parthenocarpy of a plant.

Examples of the parthenocarpy regulatory gene of the present invention encompass a gene derived from tomato (SEQ ID NOs: 2 and 6 (ORF), and SEQ ID NO: 11 (full-length cDNA), SEQ ID NO: 16 (a gene on genomic DNA containing an intron)), a gene derived from S. pimpinellifolium (wild relative of tomato, SEQ ID NO: 18) or from S. peruvianum (wild relative of tomato, SEQ ID NO: 20), a gene derived from S. tuberosum phureja (potato, SEQ ID NO: 22), and a gene derived from S. melongena (eggplant, SEQ ID NO: 24). Moreover, the parthenocarpy regulatory gene of the present invention encompasses a gene including nucleotides which has a parthenocarpy regulation activity and has a sequence identity of 70% or higher, 75% or higher, 80% or higher, 85% or higher, 90% or higher, 95% or higher, 96% or higher, 97% or higher, 98% or higher, or 99% or higher, relative to a base sequence of the above exemplified polynucleotide.

[2. Polypeptide as Parthenocarpy Regulation Protein]

The polypeptide of the present invention is a product obtained by translating the parthenocarpy regulatory gene described in the part [1. Parthenocarpy regulatory gene] above and has at least an activity to regulate parthenocarpy of a plant. As above described, the polypeptide of the present invention inhibits appearance of the trait of parthenocarpy, i.e., negatively regulated.

The polypeptide of the present invention can be isolated from a natural resource or can be chemically synthesized. Specifically, the polypeptide encompasses a product obtained by purifying a substance isolated from a natural resource, a product obtained in chemical synthesis process, and a translated product obtained from a procaryotic host or a eucaryotic host (e.g., bacterial cell, yeast cell, higher plant cell, insect cell, or mammalian cell) by a recombination technique.

Specifically, the polypeptide of the present invention is a polypeptide described in any of (1) through (4) below. Note that the polypeptide is a translated product of the parthenocarpy regulatory gene. Therefore, a meaning of the wordings such as “hybridize under a stringent condition” can be understood with reference to the descriptions in [1. Parthenocarpy regulatory gene] above.

(1) A polypeptide having an amino acid sequence represented by SEQ ID NO: 1.
(2) A polypeptide (i) having a sequence identity of 70% or higher relative to the amino acid sequence represented by SEQ ID NO: 1 and (ii) having a parthenocarpy regulation activity. Note that the sequence identity is preferably 75% or higher or 80% or higher, more preferably 85% or higher, more preferably 90% or higher, further preferably 95% or higher, particularly preferably 96% or higher, 97% or higher, 98% or higher, or 99% or higher.
(3) A polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity. Note that the number of amino acids which are substituted, deleted, inserted, and/or added is preferably 1 to 72; or 1 to 58, more preferably 1 to 43, more preferably 1 to 29, further preferably 1 to 14, particularly preferably 1 to 5 or 6. (4) A polypeptide that (i) is encoded by a polynucleotide which is hybridized, under a stringent condition, with a polynucleotide that has a sequence complementary to a polynucleotide for encoding the polypeptide described in the above (1) and (ii) has a parthenocarpy regulation activity.

The polypeptide may be a polypeptide constituted by bonding of amino acids. Note, however, that the polypeptide is not limited to this and can contain a structure other than a polypeptide structure. The structure other than polypeptide can be a sugar chain, an isoprenoid group, and the like, but is not limited to these in particular.

Note that examples of the polypeptide of the present invention as a parthenocarpy regulation protein encompass a polypeptide derived from tomato (SEQ ID NO: 1), a polypeptide derived from S. pimpinellifolium (wild relative of tomato, SEQ ID NO: 19), a polypeptide derived from S. peruvianum (wild relative of tomato, SEQ ID NO: 21), a polypeptide derived from S. tuberosum phureja (potato, SEQ ID NO: 23), and a polypeptide derived from S. melongena (eggplant, SEQ ID NO: 25).

[3. Recombinant Expression Vector and Transformant]

The present invention also provides (i) a recombinant expression vector in which the parthenocarpy regulatory gene of the present invention is incorporated such that the parthenocarpy regulatory gene can be expressed and (ii) a transformant in which the recombinant expression vector or the parthenocarpy regulatory gene is introduced such that the recombinant expression vector or the parthenocarpy regulatory gene can be expressed.

A type of a vector for constituting the recombinant expression vector is not limited to a particular one, and a vector which can be expressed in a host cell can be selected as appropriate. That is, a promoter sequence is appropriately selected depending on a type of a host cell, and a vector, in which the promoter sequence and the parthenocarpy regulatory gene of the present invention are incorporated in, for example, a plasmid, a phagemid, a cosmid, or the like, can be used as an expression vector.

The transformant encompasses an organism, as well as a cell, a tissue, and an organ in which the recombinant expression vector or the parthenocarpy regulatory gene is introduced such that the recombinant expression vector or the parthenocarpy regulatory gene can be expressed. A species of the transformant is not limited to a particular one and can be, for example, a microorganism such as E. coli, a plant, an animal, or the like.

[4. Method for Evaluating Parthenocarpy of Plant]

A method of the present invention for evaluating parthenocarpy includes a step of checking (i) whether or not expression of the parthenocarpy regulatory gene is inhibited in a plant or (ii) whether or not a function of a polypeptide encoded by the parthenocarpy regulatory gene is inhibited in the plant. Here, the “parthenocarpy regulatory gene” indicates the parthenocarpy regulatory gene described in [1. Parthenocarpy regulatory gene] above, and the “polypeptide encoded by the parthenocarpy regulatory gene” indicates the polypeptide described in [2. Polypeptide as parthenocarpy regulation protein] above. Note that the inhibition of expression of the parthenocarpy regulatory gene encompasses inhibition of gene transcription and inhibition of translation into a protein.

Moreover, in the checking step, it is preferable to select, as a candidate plant whose parthenocarpy is regulated, (i) a plant in which expression of the parthenocarpy regulatory gene is inhibited or (ii) a plant in which the function of the polypeptide encoded by the parthenocarpy regulatory gene is inhibited.

A method for carrying out the checking step is not limited to a particular one and can be, for example, any of methods described in the following 1) through 3). Among these methods, the method of 1) or 2) is preferable in view of easiness in working.

1) An expression level of the parthenocarpy regulatory gene in a target plant is measured and, if necessary, compared with a standard gene expression level, and thus whether or not expression of the parthenocarpy regulatory gene is inhibited is examined. The expression level of the gene can be measured by, for example, (i) measuring an amount of a transcript with a method such as quantitative RT-PCR or quantitative real time PCR or (ii) measuring an amount of a translated product with a method such as quantitative Western blotting. The standard gene expression level indicates, for example, an expression level of the parthenocarpy regulatory gene in a conspecific plant that does not show parthenocarpy (e.g., an individual having the parthenocarpy regulatory gene as a homozygote). Note that, in a case where transcription or translation of the parthenocarpy regulatory gene is substantially completely inhibited with the use of a method such as gene disruption or RNAi, the comparison with the standard gene expression level may be unnecessary.
2) A base sequence of a parthenocarpy regulatory gene (genomic DNA, mRNA, or cDNA) of a target plant is analyzed. Then, in a case where a mutation(s) that influences a function of the polypeptide encoded by the parthenocarpy regulatory gene is found as a result of the analysis, it is determined that the function of the polypeptide is inhibited. The mutation(s) that influences the function of the polypeptide can be, for example, defect of several tens to several thousands of base pairs of nucleotides including a coding region of the parthenocarpy regulatory gene, preferably defect of at least 100 bp to 1100 bp of nucleotides, or defect of at least approximately 270 bp to 1100 bp nucleotides. Note that such a mutation(s) may occur in at least one of two parthenocarpy regulatory genes (i.e., a pair of parthenocarpy regulatory genes) in a homologous chromosome, and it is preferable that the mutation(s) occurs in both the two parthenocarpy regulatory genes (i.e., recessive homozygote).
Moreover, in a case where a marker sequence that shows a linkage with the mutation(s) on the parthenocarpy regulatory gene is utilized, the base sequence of the parthenocarpy regulatory gene is to be directly or indirectly checked, and it is thus possible to carry out the checking step. The marker sequence is, for example, located within approximately 5.5 cM (i.e., approximately 5.5 cM to 0 cM), preferably located within approximately 5.3 cM, more preferably located within approximately 1.2 cM, further preferably located within approximately 1.1 cM, from a location of the parthenocarpy regulatory gene. Note that it is of course possible to use the mutation(s) on the parthenocarpy regulatory gene as a marker sequence.
3) A polypeptide encoded by the parthenocarpy regulatory gene is isolated from a target plant, and an amino acid sequence of the polypeptide is analyzed. Then, in a case where a mutation(s) (e.g., generation of a truncated body that causes deletion of several tens or more of amino acids), which influences a function of the polypeptide, is found as a result of the analysis, it is determined that the function of the polypeptide is inhibited.

A type of a plant, to which the evaluating method of the present invention is applied, is not limited to a particular one, and is preferably solanaceous plants such as tomato, eggplant, pimiento, paprika, pepper, and potato, more preferably solanum plants such as tomato, eggplant, and potato, further preferably tomato or eggplant, particularly preferably tomato. Moreover, examples of a plant to which the evaluating method is applied encompass a breeding material (parental plant, i.e., pollen parent or seed parent) and offspring obtained by breeding. Note that the term “breeding” indicates a concept that encompasses (i) a method utilizing crossing and (ii) a genetic engineering method.

The phenotypic expression of parthenocarpy sometimes becomes unstable depending on a cultivation condition or a cultivar or line and therefore it is often difficult to evaluate based only on appearance of a plant. Moreover, a plant in which the parthenocarpy regulatory gene is heterozygous is worth using as a breeding material or the like. In particular, when the parthenocarpy regulatory gene is heterozygous, it is sometimes impossible to clearly only from appearance of fruit phenotype whether or not the parthenocarpy can be shown. The method of the present invention for evaluating the parthenocarpy is carried out at a gene level, and therefore trait evaluation can be carried out without making genes homozygous. For example, this makes it possible to significantly improve efficiency in breeding selection. Further, the parthenocarpy regulatory gene has been identified, and it is therefore possible to segregate, with the use of a gene engineering techniques or the like, linkage with an unfavorable trait which has naturally cosegregated with the parthenocarpy regulatory gene.

[5. Method for Producing Parthenocarpy-Regulated Plant, and Produced Plant]

A form (hereinafter, referred to as “form 1”) of a method of the present invention for producing a parthenocarpy-regulated plant is a method that includes a step of selecting a parthenocarpy-regulated plant by performing the evaluating method described in [4. Method for evaluating parthenocarpy of plant] above.

Alternatively, another form (hereinafter, referred to as “form 2”) of the method of the present invention for producing a parthenocarpy-regulated plant is a method that includes a step of inhibiting expression of the parthenocarpy regulatory gene in the plant or a step of inhibiting a function of a polypeptide encoded by the parthenocarpy regulatory gene in the plant. Here, the “parthenocarpy regulatory gene” is the one described in [1. Parthenocarpy regulatory gene] above, and the “polypeptide encoded by the parthenocarpy regulatory gene” is the one described in [2. Polypeptide as parthenocarpy regulation protein] above.

Note that, in the form 2, it is preferable to inhibit expression of the parthenocarpy regulatory gene by disrupting the parthenocarpy regulatory gene or introducing, to the plant, a polynucleotide that inhibits expression of the parthenocarpy regulatory gene. Note that the wording “inhibit expression of the parthenocarpy regulatory gene” encompasses (i) inhibition of transcription of a gene and (ii) inhibition of translation into protein.

A method for disrupting the parthenocarpy regulatory gene is not limited to a particular one. For example, it can be performed (i) by a specific gene disruption using homologous recombination, (ii) by introducing a termination codon to the parthenocarpy regulatory gene using site-directed mutagenesis, and (iii) by a partial physical gene disruption using irradiation of a heavy ion beam or the like.

Moreover, the method for introducing, to the plant, a polynucleotide that inhibits expression of the parthenocarpy regulatory gene is not limited to a particular one and can be, for example, a method such as RNA interference or antisense RNA. A method for introducing, to the plant, an expression cassette (e.g., vector) containing the polynucleotide is not limited to a particular one, and it is possible to employ, as appropriate, a method using polyethyleneglycol, electroporation, a method via Agrobacterium, or a method using a particle gun. Note that the expression cassette containing the polynucleotide can be introduced to a plant cell, a callus, a plant tissue, or a plant individual.

Note that expression of the parthenocarpy regulatory gene can be inhibited by introducing a mutation(s), which inhibits gene expression, to an expression adjusting sequence (e.g., a promoter sequence or an enhancer sequence) that regulates expression of the parthenocarpy regulatory gene.

The present invention also provides a parthenocarpy-regulated plant, which has been produced by the above described production method. The term “parthenocarpy-regulated plant” indicates a concept that encompasses (i) a plant in which the trait of parthenocarpy is shown and (ii) a plant in which the trait of parthenocarpy is not shown but which has a predisposition of the parthenocarpy.

A type of a plant which is to be produced by the method of the present invention is not limited to a particular one and is preferably solanaceous plants such as tomato, eggplant, pimiento, paprika, pepper, and potato, more preferably solanum plants such as tomato, eggplant, and potato, further preferably tomato or eggplant, particularly preferably tomato.

[6. Example of Concrete Aspect of Present Invention]

That is, the present invention encompasses any of the following 1) through 11):

1) A method for evaluating parthenocarpy of a plant, the method including the step of: checking whether or not expression of a parthenocarpy regulatory gene, which includes a polynucleotide described in any of (1) through (4) below, is inhibited in the plant; or checking whether or not a function of a polypeptide, which is encoded by the parthenocarpy regulatory gene, is inhibited in the plant. According to a form of the checking step, for example, it is checked, in at least a bud of the plant, (i) whether or not expression of the parthenocarpy regulatory gene is inhibited or (ii) whether or not the function of the polypeptide encoded by the parthenocarpy regulatory gene is inhibited.
(1) A polynucleotide that encodes a polypeptide having an amino acid sequence represented by SEQ ID NO: 1, (2) a polynucleotide that encodes a polypeptide (i) having a sequence identity of 70% or higher relative to the amino acid sequence represented by SEQ ID NO: 1 and (ii) having a parthenocarpy regulation activity, (3) a polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and (4) a polynucleotide that (i) is hybridized with a polynucleotide, which has a sequence complementary to the polynucleotide described in the above (1), under a stringent condition and (ii) encodes a polypeptide having a parthenocarpy regulation activity.
2) The method described in the above 1) in which, in the step of checking, a base sequence of the parthenocarpy regulatory gene or an expression level of the parthenocarpy regulatory gene is checked.
3) The method described in the above 1) or 2) in which, in the step of checking, a plant in which the expression of the parthenocarpy regulatory gene is inhibited or a plant in which the function of the polypeptide encoded by the parthenocarpy regulatory gene is inhibited is selected as a parthenocarpic plant.
4) A method for producing a parthenocarpy-regulated plant, the method including the step of selecting a parthenocarpic plant by carrying out the method described in any one of the above 1) through 3).
5) A method for producing a parthenocarpy-regulated plant, the method including the step of: inhibiting, in a plant, gene expression of a parthenocarpy regulatory gene that includes a polynucleotide described in any of (1) through (4) below; or inhibiting, in a plant, a function of a polypeptide encoded by the parthenocarpy regulatory gene. According to an aspect of the step, for example, in at least a bud of the plant, gene expression of the parthenocarpy regulatory gene is inhibited or the function of the polypeptide encoded by the parthenocarpy regulatory gene is inhibited.
(1) A polynucleotide that encodes a polypeptide having an amino acid sequence represented by SEQ ID NO: 1, (2) a polynucleotide that encodes a polypeptide (i) having a sequence identity of 70% or higher relative to the amino acid sequence represented by SEQ ID NO: 1 and (ii) having a parthenocarpy regulation activity, (3) a polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and (4) a polynucleotide that (i) is hybridized with a polynucleotide, which has a sequence complementary to the polynucleotide recited in the above (1), under a stringent condition and (ii) encodes a polypeptide having a parthenocarpy regulation activity.
6) The method described in the above 5) in which, in the step, (i) a polynucleotide, which inhibits the gene expression of the parthenocarpy regulatory gene, is introduced into the plant or (ii) the parthenocarpy regulatory gene is disrupted.
7) The method described in any of the above 1) through 6) in which the plant is a solanaceous plant.
8) The method described in the above 7) in which the plant is a tomato.
9) A parthenocarpy-regulated plant produced by the method described in any of the above 4) through 6).
10) A parthenocarpy regulatory gene that includes a polynucleotide described in any of (1) through (4) below. According to a form of the parthenocarpy regulatory gene, for example, the plant shows parthenocarpy by inhibiting gene expression of the parthenocarpy regulatory gene in at least a bud of the plant. (1) A polynucleotide that encodes a polypeptide having an amino acid sequence represented by SEQ ID NO: 1, (2) a polynucleotide that encodes a polypeptide (i) having a sequence identity of 70% or higher relative to the amino acid sequence represented by SEQ ID NO: 1 and (ii) having a parthenocarpy regulation activity, (3) a polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and (4) a polynucleotide that (i) is hybridized with a polynucleotide, which has a sequence complementary to the polynucleotide described in the above (1), under a stringent condition and (ii) encodes a polypeptide having a parthenocarpy regulation activity.
11) A polypeptide described in any of (1) through (4) below. According to a form of the polypeptide, for example, the plant shows parthenocarpy by inhibiting a function of the polypeptide in at least a bud of the plant.
(1) A polypeptide having an amino acid sequence represented by SEQ ID NO: 1, (2) a polypeptide (i) having a sequence identity of 70% or higher relative to the amino acid sequence represented by SEQ ID NO: 1 and (ii) having a parthenocarpy regulation activity, (3) a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and (4) a polypeptide that (i) is encoded by a polynucleotide which is hybridized, under a stringent condition, with a polynucleotide that has a sequence complementary to a polynucleotide for encoding the polypeptide described in the above (1) and (ii) has a parthenocarpy regulation activity.

According to another aspect, the present invention can be any of the following 12) through 17):

12) A method for evaluating parthenocarpy of a plant, the method including the step of: checking whether or not gene expression of a parthenocarpy regulatory gene, which includes a polynucleotide described in any of (S1) through (S4) below, is inhibited in the plant; or checking whether or not a function of a polypeptide, which is encoded by the parthenocarpy regulatory gene, is inhibited in the plant. According to a form of the checking step, for example, it is checked, in at least a bud of the plant, (i) whether or not gene expression of the parthenocarpy regulatory gene is inhibited or (ii) whether or not the function of the polypeptide encoded by the parthenocarpy regulatory gene is inhibited.
(S1) A polynucleotide that encodes a polypeptide having an amino acid sequence represented by SEQ ID NO: 25, (S2) a polynucleotide that encodes a polypeptide (i) having a sequence identity of 70% or higher relative to the amino acid sequence represented by SEQ ID NO: 25 and (ii) having a parthenocarpy regulation activity, (S3) a polynucleotide that encodes a polypeptide (i) having an amino acid sequence in which 1 to 87 amino acids are substituted in, deleted from, inserted into, and/or added to the amino acid sequence represented by SEQ ID NO: 25 and (ii) having a parthenocarpy regulation activity, and (S4) a polynucleotide that (i) is hybridized with a polynucleotide, which has a sequence complementary to the polynucleotide described in the above (S1) under a stringent condition and (ii) encodes a polypeptide having a parthenocarpy regulation activity.
13) A method for producing a parthenocarpy-regulated plant, the method including the step of selecting a parthenocarpic plant by carrying out the method described in the above 12).

14) A method for producing a parthenocarpy-regulated plant, the method including the step of: inhibiting, in a plant, gene expression of a parthenocarpy regulatory gene that includes a polynucleotide described in any of (S1) through (S4) below; or inhibiting, in a plant, a function of a polypeptide encoded by the parthenocarpy regulatory gene. According to a form of the step, for example, in at least a bud of the plant, gene expression of the parthenocarpy regulatory gene is inhibited or the function of the polypeptide encoded by the parthenocarpy regulatory gene is inhibited.

(S1) A polynucleotide that encodes a polypeptide having an amino acid sequence represented by SEQ ID NO: 25, (S2) a polynucleotide that encodes a polypeptide (i) having a sequence identity of 70% or higher relative to the amino acid sequence represented by SEQ ID NO: 25 and(ii) having a parthenocarpy regulation activity, (S3) a polynucleotide that encodes a polypeptide (i) having an amino acid sequence in which 1 to 87 amino acids are substituted in, deleted from, inserted into, and/or added to the amino acid sequence represented by SEQ ID NO: 25 and (ii) having a parthenocarpy regulation activity, and (S4) a polynucleotide that (i) is hybridized with a polynucleotide, which has a sequence complementary to the polynucleotide described in the above (S1) under a stringent condition and (ii) encodes a polypeptide having a parthenocarpy regulation activity.

15) A parthenocarpy-regulated plant produced by the method described in the above 13) or 14). 16) A parthenocarpy regulatory gene that includes a polynucleotide described in any of (S1) through (S4) below. According to a form of the parthenocarpy regulatory gene, for example, the plant shows parthenocarpy by inhibiting gene expression of the parthenocarpy regulatory gene in at least a bud of the plant. (S1) A polynucleotide that encodes a polypeptide having an amino acid sequence represented by SEQ ID NO: 25, (S2) a polynucleotide that encodes a polypeptide (i) having a sequence identity of 70% or higher relative to the amino acid sequence represented by SEQ ID NO: 25 and (ii) having a parthenocarpy regulation activity, (S3) a polynucleotide that encodes a polypeptide (i) having an amino acid sequence in which 1 to 87 amino acids are substituted in, deleted from, inserted into, and/or added to the amino acid sequence represented by SEQ ID NO: 25 and (ii) having a parthenocarpy regulation activity, and (S4) a polynucleotide that (i) is hybridized with a polynucleotide, which has a sequence complementary to the polynucleotide described in the above (S1) under a stringent condition and (ii) encodes a polypeptide having a parthenocarpy regulation activity.

17) A polypeptide described in any of (S1) through (S4) below. According to a form of the polypeptide, for example, the plant shows parthenocarpy by inhibiting a function of the polypeptide in at least a bud of the plant.
(S1) A polypeptide having an amino acid sequence represented by SEQ ID NO: 25, (S2) a polypeptide (i) having a sequence identity of 70% or higher relative to the amino acid sequence represented by SEQ ID NO: 25 and (ii) having a parthenocarpy regulation activity, (S3) a polypeptide (i) having an amino acid sequence in which 1 to 87 amino acids are substituted in, deleted from, inserted into, and/or added to the amino acid sequence represented by SEQ ID NO: 25 and (ii) having a parthenocarpy regulation activity, (S4) a polypeptide that (i) is encoded by a polynucleotide which is hybridized, under a stringent condition, with a polynucleotide that has a sequence complementary to a polynucleotide for encoding the polypeptide described in the above (S1) and (ii) has a parthenocarpy regulation activity.
Note that, in the above 12) through 17), the sequence identity of the amino acid sequence is preferably 75% or higher or 80% or higher, more preferably 85% or higher, more preferably 90% or higher, further preferably 95% or higher, particularly preferably 96% or higher, 97% or higher, 98% or higher, or 99% or higher.

The present invention is not limited to the embodiments, but can be altered by a skilled person in the art within the scope of the claims. That is, an embodiment derived from a proper combination of technical means appropriately modified within the scope of the claims is also encompassed in the technical scope of the present invention.

### EXAMPLES

[1. Identification of Tomato-Derived Parthenocarpy Responsible Gene A]

[Breeding of Genetic Analysis Population]

By crossing a parthenocarpic tomato line LS935 (obtained from National Institute of Vegetable and Tea Science) and a non-parthenocarpic tomato line Saladette (LA2662) (obtained from Tomato Genetics Resource Center in US), a population of F₂ individuals was obtained. With the use of the F₂ population, a tomato linkage map in which mapped parthenocarpy responsible gene A was constructed and genetic analysis of parthenocarpy was carried out as described below. Further, a selfed progeny F₃ population, which was a line selected from the F₂ population, was used in the detailed genetic analysis of parthenocarpy described below.

Note that the tomato line LS935 is a genetic resource obtained by introducing, as a backcrossed line, Moneymaker (which is a large fruit tomato cultivar) into Severianin which is a cultivar that reportedly shows parthenocarpy. Saladette is a genetic resource introduced as a large fruit tomato line and shows non-parthenocarpy (i.e., does not show parthenocarpy).

[Method for Evaluating Parthenocarpy]

A stigma was removed from flower before anthesis, and mature fruit weight of stigma-removed fruit was measured, and presence or absence of a seed in stigma-removed fruit was confirmed. A weight of each of the mature stigma-removed fruits, which had 3 or more flower clusters per plant, was measured, and a weight of each of mature open-pollinated fruits, which had 1 or more flower cluster(s) per plant, was measured. The weights of the stigma-removed fruits were compared with the weights of the open-pollinated fruits, and a line, from which a mature fruit having a fruit weight similar to that of the open-pollinated fruits was obtained, was determined to have parthenocarpy. Note that, with regard to lines other than the RNAi transgenic tomato, it is difficult to evaluate parthenocarpy when the open-pollinated fruit is small and a difference from the stigma-removed fruit is small. In such a case, parthenocarpy was evaluated again using propagated seedlings.

[Construction of Linkage Map]

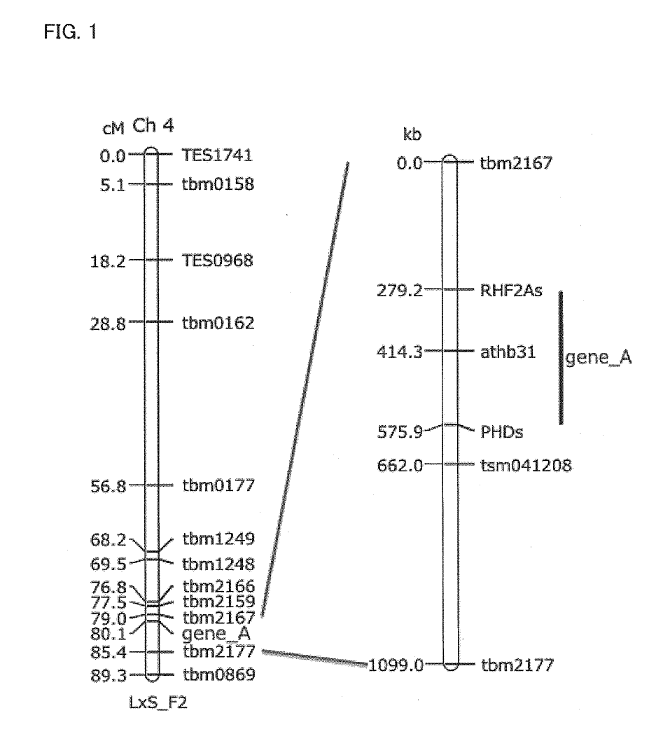

With the use of reported SSR (simple sequence repeat) markers (Reference Literature: Ohyama A. et al. (2009) Mol. Breed. 23: 685-691, Shirasawa L. et al. (2010) Theor. Appl. Genet. 121: 731-739.) and markers (which is being developed by the applicant) of SSR in the neighboring of reported SNP markers, a linkage map illustrated in FIG. 1 was constructed, and parthenocarpy/non-parthenocarpy traits were evaluated using F₂ population including 100 individuals and mapped on the linkage map. In the construction of the linkage map, Mapmaker exp 3.0 was used. Note that a left part of FIG. 1 is a linkage map of chromosome 4 of tomato. In FIG. 1, names of markers that begin with “TES” indicate reported SSR markers, and names of markers that begin with “tbm” indicate markers originally developed by the applicant.

[Detailed Genetic Analysis of Parthenocarpy]

From the genetic analysis with the use of the F₂ population, it has been found that the parthenocarpy responsible gene A is located at chromosome 4 of tomato and is located in a region of approximately 5 cM between two SSR markers (see FIG. 1). As a result of comparative analysis between nucleotide sequence of the SSR markers and tomato genome sequence (sol genomics network http://solgenomics.net/), a physical distance between the two SSR markers corresponded to approximately 1100 kb.

Next, with the use of the tomato genome sequence, several SSRs of the region in which the parthenocarpy responsible gene A is located were newly extracted and polymorphism was searched. Moreover, SNP polymorphisms in the neighboring of a candidate gene that was located in the region were newly searched. Some F₂ individuals, whose genotype in the region was heterozygous, were selected from the F₂ population, and genotype of progeny F₃ individuals was determined. Thus, genetic recombinated individuals were selected, and parthenocarpy of the individual was evaluated. As a result of the analysis, a locus of the parthenocarpy responsible gene A was narrowed down to approximately 300 kb region between two SNP markers. Note that genetic linkage markers located on neighboring of approximately 300 kb region are shown in a right part of FIG. 1. RHF2As and PHDs are newly developed SNP markers, tsm041208 is a newly developed SSR marker, and athb31 is a newly developed insertion-deletion mutation marker.

For the non-parthenocarpic line “Saladette” and the parthenocarpic line “LS935”, a base sequence of the candidate region of approximately 300 kb was determined, and polymorphism between two lines was searched. As a result, by comparing with the genomic sequence (SEQ ID NO: 3) of “Saladette”, a deletion mutation (SEQ ID NO: 5) of 1034 bp was found in the genomic sequence (SEQ ID NO: 4) of “LS935”.

The deletion mutation existed on a putative gene that has open reading frame as represented by SEQ ID NO: 6, and it was estimated that a part of exon 1 and a part of intron 1 of the putative gene were lost from a genome of “LS935”. Under the circumstances, it was predicted that the deletion mutation of the putative gene that had the open reading frame represented by SEQ ID NO: 6 induced parthenocarpy, and the following experiment was carried out. Note that a base sequence (including an intron) on the genome of the putative gene of “Saladette” is represented by SEQ ID NO: 16, and a corresponding base sequence of the genome of “LS935” is represented by SEQ ID NO: 17.

[Obtain Full-Length cDNA Sequence of Parthenocarpy Responsible Gene A]

From aboveground tissues including leaves, stems, and flowers of a non-parthenocarpic tomato cultivar “Shugyoku”, total RNA was extracted by a Trizol method, and cDNA was synthesized with the use of SMARTer RACE cDNA amplification Kit (manufactured by Takara Bio Inc.). By using the cDNA as a template, amplification of cDNA including a transcription start point and a translation start point of mRNA was attempted with the use of a 5′-RACE method in which the polynucleotide represented by SEQ ID NO: 7 was used as a gene-specific primer. An amplified product was cloned into an E. coli vector, and nucleotide sequence thereof was determined by Sanger's method. As a result, a sequence of 842 bp at a 5′ end (including exon 1 (i.e., 298 bp of 5′-UTR (untranslated region) and 541 bp of 5′ end of ORF) and 3 bp of exon 2) of full-length cDNA was obtained.

Similarly, with a 3′-RACE method in which the polynucleotide represented by SEQ ID NO: 8 was used as a gene-specific primer, (i) amplification of cDNA including a translation end point and a polyadenylation region, (ii) cloning to an E. coli vector, and (iii) determination of a nucleotide sequence were attempted. As a result, a sequence of 539 bp (including 305 bp of exon 2 (i.e., 3′ end of ORF (including a translation end point)) and 234 bp of 3′-UTR) at a 3′ end of full-length cDNA was obtained.

Further, with the use of a pair of primers, i.e., a primer represented by SEQ ID NO: 9 and a primer represented by SEQ ID NO: 10, cDNA was amplified by PCR which was carried out using, as a template, cDNA identical with the above described one. Then, nucleotide sequence of the cDNA thus amplified was determined by direct sequencing. Then, a base sequence (represented by SEQ ID NO: 11) of 1405 bp was obtained by combining the 5′ end region and the 3′ end region (which were obtained above) and the other regions of the full-length cDNA based on overlaps of sequences, and thus the base sequence represented by SEQ ID NO: 11 was obtained as a full-length cDNA sequence of the parthenocarpy responsible gene A.

[Analysis of Amino Acid Sequence of Parthenocarpy Responsible Gene A and Search for Homologue Gene]

As a result of blast search (http://blast.ncbi.nlm.nih.gov/), it was found that the parthenocarpy responsible gene A belongs to a Zn finger homeodomain type homeobox gene in which (i) a part from 56th valine to 112th asparagine in an amino acid sequence to be encoded was predicted to be a Zn finger domain and (ii) a part from 219th lysine to 276th asparagine in the amino acid sequence to be encoded was predicted to be a homeo domain. The Zn finger homeodomain type homeobox gene is predicted to regulate morphogenesis as a transcription factor. Moreover, homologs of the parthenocarpic gene A were found in databases, that is, an S. pimpinellifolium (wild relative of tomato)-derived homolog, an S. peruvianum (wild relative of tomato)-derived homolog, an S. tuberosum phureja (potato)-derived homolog, and an S. melongena (eggplant)-derived homolog were found. Homologies of amino acid sequences of the respective homologs relative to the amino acid sequence which is encoded by the parthenocarpic gene A were as follows: that is, a homology of the S. pimpinellifolium (wild relative of tomato)-derived homologue (SEQ ID NO: 19) was 95%, a homology of the S. peruvianum (wild relative of tomato)-derived homologue (SEQ ID NO: 21) was 94%, a homology of the S. tuberosum phureja (potato)-derived homologue (SEQ ID NO: 23) was 84%, and a homology of the S. melongena (eggplant)-derived homologue (SEQ ID NO: 25) was 75%. Note that the database used to search the homolog gene of eggplant was prepared by the inventors on their own and is not yet open to the public.

[Isolation of Promoter Region of Parthenocarpy Responsible Gene A]

The full-length cDNA sequence of the parthenocarpy responsible gene A represented by SEQ ID NO: 11 corresponds to a part from nucleotide number 9421 to nucleotide number 11603 in the genomic sequence represented by SEQ ID NO: 3. Therefore, an upstream genomic sequence thereof is presumed to be a promoter region that relates to control of expression of the parthenocarpy responsible gene A. Under the circumstances, PCR primers for amplifying the region was synthesized, and PCR was carried out with the use of the PCR primers and genomic DNA of a tomato cultivar “Momotaro 8” as a template. An obtained PCR product was digested by restricted enzymes HindIII and SmaI, and cloned by being inserted into a HindIII-Sural site of a cloning vector pHSG398.

Then, a nucleotide sequence of an obtained clone was confirmed by the Sanger's method and was compared with the sequence represented by SEQ ID NO: 3. Thus, a clone was selected which did not contain a mutation(s) caused by the PCR amplification. The clone thus obtained had 2572 bp and had a genome-derived HindIII recognition site at a 5′ end, and a PCR primer-derived SmaI recognition site at a 3′ end. Specifically, the clone included (i) 2239 bp in a part upstream to a transcription start point of the parthenocarpy responsible gene A, (ii) 327 bp from the transcription start point to a part of a protein coding region via 5′-UTR and a translation start point, and (iii) 6 bp of the PCR primer-derived SmaI recognition site. This DNA fragment (SEQ ID NO: 12) was used in the following experiment as a promoter of the parthenocarpy responsible gene A.

[Construction of RNA Interference (RNAi) Induction Vector]

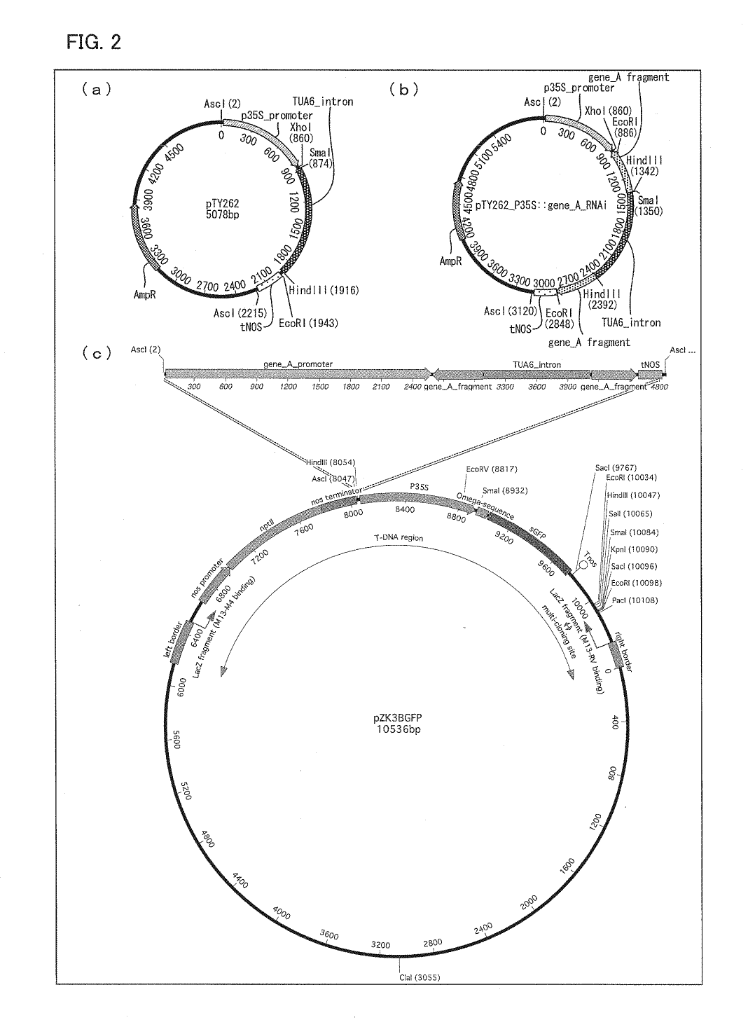

With the use of primers respectively represented by SEQ ID NO: 13 and SEQ ID NO: 14, PCR was carried out using cDNA of the tomato cultivar “Shugyoku” as a template, and thus 500 bp of DNA fragment represented by SEQ ID NO: 15 was obtained. The fragment had a SmaI recognition site and a HindIII recognition site at a 5′ end, and an EcoRI recognition site and an XhoI recognition site at a 3′ end. The fragment was inserted into a cloning vector pTY262 (AB736152 (DDBJ)), and RNAi induction chimeric gene with respect to the parthenocarpy responsible gene A was constructed. The pTY262 vector had an expression cassette including a CaMV35S promoter and a Nos terminator. In the pTY262 vector, cloning sites of exogenous sequences are arranged so that intron 1 of a tomato tubulin gene is located between the cloning sites of exogenous sequences (see (a) of FIG. 2).

First, the above described 500 bp fragment (SEQ ID NO: 15) was digested by HindIII and EcoRI and inserted ahead of the Nos terminator (tNos). Then, the above described 500 bp fragment was digested by SmaI and XhoI and inserted behind the CaMV35S promoter of the obtained vector. An RNAi induction chimeric gene thus obtained had a structure in which the substantially entire 500 bp fragment represented by SEQ ID NO: 15 was arranged in an inverted repeat sequence into which the intron 1 of the tomato tubulin gene was inserted. Expression of the RNAi induction chimeric gene was induced by the CaMV35S promoter which induces constitutive expression of general plants (see (b) of FIG. 2). Further, an RNAi induction chimeric gene was constructed by replacing the CaMV35S promoter with a promoter of the parthenocarpy responsible gene A represented by SEQ ID NO: 12. Next, a clone having the RNAi induction chimeric gene thus constructed was digested by AscI, and pZK3BGFP_geneA-RNAi (see (c) of FIG. 2) was constructed by inserting a fragment including the obtained RNAi induction chimeric gene into an AscI recognition site of a modified binary vector pZK3BGFP in which a reporter gene including a CaMV35S promoter, GFP, and a Nos terminator was inserted into a binary vector pZK3B (Reference Literature: Kuroda et al., Biosci Biotech Biochem, (2010) 74: 2348-2351).

[Production of RNAi Transgenic Tomato]

Transformation of tomato was carried out with the use of the RNAi induction chimeric gene (vector) illustrated in (c) of FIG. 2. Specifically, with the use of an Agrobacterium method (Sun et al. 2006 Plant Cell Physiol 47: 426-431), cotyledon of the tomato cultivar “Shugyoku” was infected with Agrobacterium having the binary vector, and cultured on kanamycin-containing medium. Then, selection was carried out and thus regenerated plant was obtained. Insertion of the gene in the regenerated plant was confirmed by PCR, and RNAi transgenic tomato was obtained. Note that it was confirmed in the RNAi transgenic tomato that expression of the parthenocarpy responsible gene A was significantly inhibited.

[Study of Parthenocarpy of RNAi Transgenic Tomato]

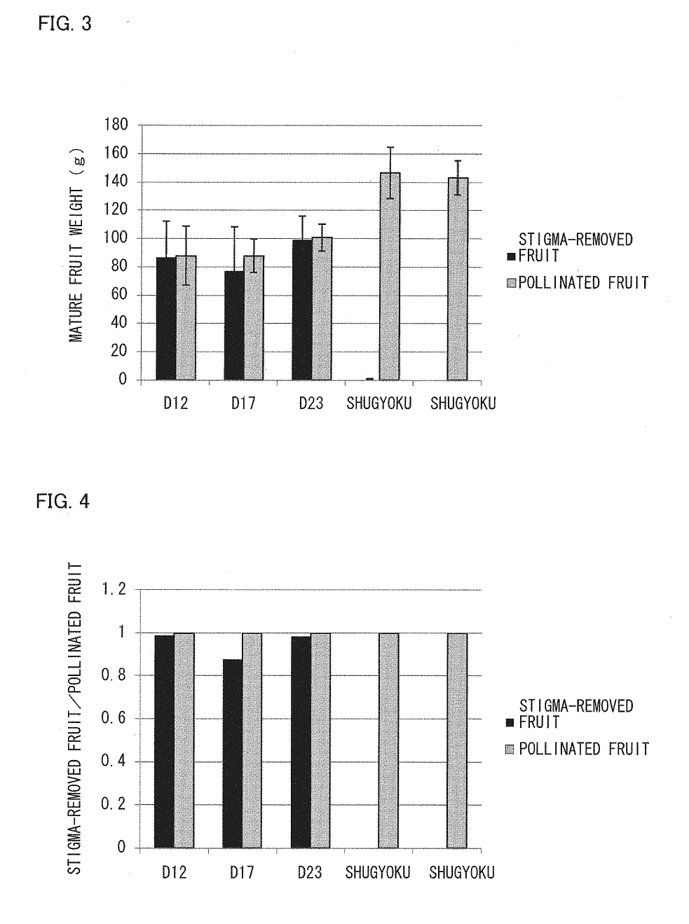

RNAi transgenic tomatoes were cultivated, and mature fruit weights of stigma-removed fruits, whose stigma had been removed before anthesis, were measured and mature fruit weights of pollinated fruits, which had been pollinated at anthesis, were measured. Moreover, non-transgenic plants were concurrently cultivated, and mature fruit weights of the non-transgenic plants were measured as controls through similar processes. Results are shown in FIGS. 3 and 4.

As shown in FIG. 3, the stigma-removed fruit of the non-transgenic tomato cultivar “Shugyoku” was not fructified and enlarged, whereas the stigma-removed fruit of the RNAi transgenic tomato was fructified and enlarged to a size similar to that of the pollinated fruit. Moreover, as shown in FIG. 4, a ratio of the fruit weight of the stigma-removed fruit of the non-transgenic tomato (i.e., the control) was nearly 0 relative to the fruit weight of the pollinated fruit which was assumed to be 1. On the other hand, a ratio of the fruit weight of the RNAi transgenic tomato was not significantly different from the ratio of the fruit weight of the pollinated fruit.

From the above result, it has been concluded that inhibition of the function (i.e., deletion mutation) of the parthenocarpy responsible gene A induces parthenocarpy.

[Analysis of Expression of Parthenocarpy Responsible Gene A]

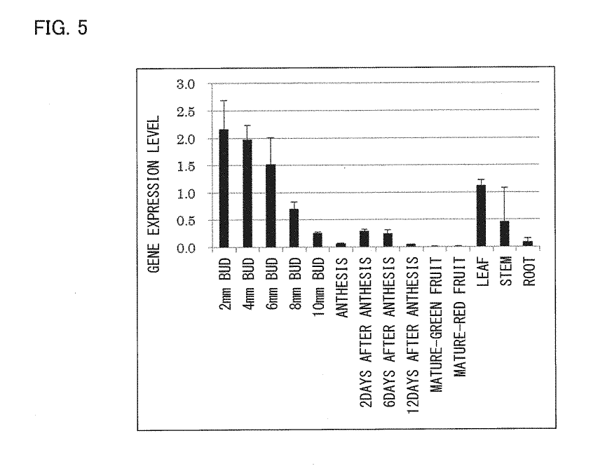

Stage-specific and tissue-specific expressions of the parthenocarpy responsible gene A were analyzed in detail by quantitative real time PCR with the use of a fluorescent real time PCR device (LC480, manufactured by Roche Diagnostics) and a LightCycler 480 SYBR Green master kit (manufactured by Roche Diagnostics). A tomato cultivar Moneymaker was used, and checked tissues are indicated in FIG. 5. Note that, in FIG. 5, “anthesis” indicates gene expression level of flowering, each of “mature-green fruit” and “mature-red fruit” indicates a gene expression level of a fruit. For amplification of the parthenocarpy responsible gene A, primers were used whose base sequences are represented by SEQ ID NO: 26 and SEQ ID NO: 27, respectively. As an endogenous standard gene for the quantitative real time PCR, a housekeeping gene Solyc04g049180.2.1 (ITAG2.30, SEQ ID NO: 28) was used which had been developed by the inventors and is constitutively-expressed in tomato tissues. For PCR amplification of the housekeeping gene, primers were used which had base sequences represented by SEQ ID NO: 29 and SEQ ID NO: 30, respectively.

As a result, it has been found that gene expression of the parthenocarpy responsible gene A shows at least 6 characteristics below (see also FIG. 5):

1. In all used tissues and stages of growth, gene expression level of the parthenocarpy responsible gene A is highest among immature buds with a total length of 2 mm.
2. The gene expression level of the parthenocarpy responsible gene A decreases as the bud grows, and decreases to 1/20 or less at anthesis.
3. Slight increase in gene expression of the parthenocarpy responsible gene A is seen during a period between the second day and the sixth day after the anthesis, but the increase level of gene expression of the gene is approximately ⅕ or less of that of the immature bud with a total length of 2 mm.
4. The expression of the parthenocarpy responsible gene A is approximately 0 in the mature-green fruit and the mature-red fruit.
5. Constant level of gene expression of the parthenocarpy responsible gene A is seen in the leaves and the stems but the gene expression level of the parthenocarpy responsible gene A is approximately ½ or less of that of the immature bud.
6. Gene expression of the parthenocarpy responsible gene A in roots is hardly seen.

[Construction of for Parthenocarpy Responsible Gene A Expression Vector]

With the use of primers having base sequences which are represented by SEQ ID NO: 31 and SEQ ID NO: 32, respectively, PCR amplification was carried out while using genomic DNA of the tomato cultivar “Shugyoku” as a template, and thus a gene fragment was obtained which had a base sequence represented by SEQ ID NO: 33. The gene fragment thus obtained corresponds to a part from the nucleotide number 7150 to the nucleotide number 12098 in the genomic sequence which is the base sequence represented by SEQ ID NO: 3. The gene fragment had primer-derived AscI recognition sites at its 5′ end and 3′ end. The gene fragment included (i) 2271 bp in a part upstream to a transcription start point of the parthenocarpy responsible gene A, (ii) 780 bp of exon 1 from the transcription start point to an intron region via 5′-UTR and a translation start point, (iii) 778 bp of an intron, (iv) 566 bp of exon 2 from immediately behind the intron to a polyA added location via a translation termination codon, and (v) 495 bp of a genomic sequence in a part downstream to the exon 2.

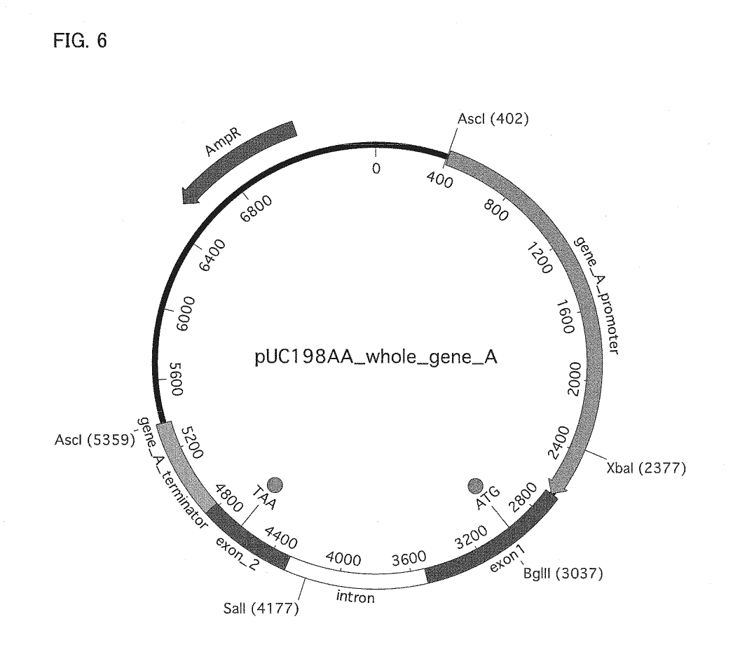

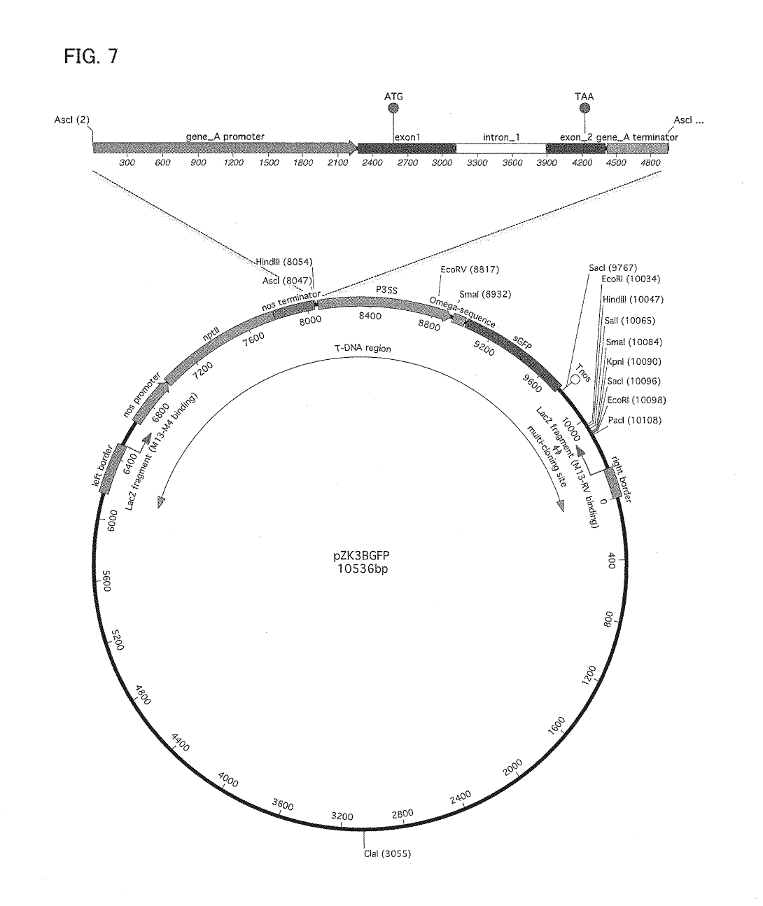

The gene fragment, which had been amplified, was digested by AscI and was then inserted into an AscI recognition site of a cloning vector pUC198AA (Reference Literature: Kuroda et al., Biosci Biotech Biochem, (2010) 74: 2348-2351) for obtaining a clone (i.e., pUC198AA_whole_gene_A in FIG. 6). A base sequence of the clone thus obtained was determined by the Sanger's method, and the base sequence was compared with the base sequence represented by SEQ ID NO: 3. Thus, a clone was selected which did not include a mutation(s) caused in PCR amplification. The clone thus selected was digested by AscI, and a binary vector pZK3BGFP_whole_Gene_A (see FIG. 7) was constructed by inserting a fragment thus obtained by digestion into an AscI recognition site of a modified binary vector pZK3BGFP in which a reporter gene including CaMV35S promoter, GFP, and a Nos terminator was inserted into a binary vector pZK3B (Kuroda et al., Biosci Biotech Biochem, (2010) 74: 2348-2351).

[Production of Transgenic Tomato in which Parthenocarpy Responsible Gene A is Expressed]

In a manner similar to that described in [Production of RNAi transgenic tomato], with the use of the Agrobacterium method, a cotyledon of a parthenocarpic tomato line LS935 was infected with Agrobacterium having a binary vector pZK3BGFP_whole_gene_A, and cultured on a kanamycin-containing medium. Then, selection was carried out and thus a regenerated plant was obtained. Insertion of the gene in the regenerated plant was confirmed by the PCR, and a transgenic tomato in which the parthenocarpy responsible gene A is expressed was obtained.

[Study of Parthenocarpy of Transgenic Tomato in which Parthenocarpy Responsible Gene A is Expressed]

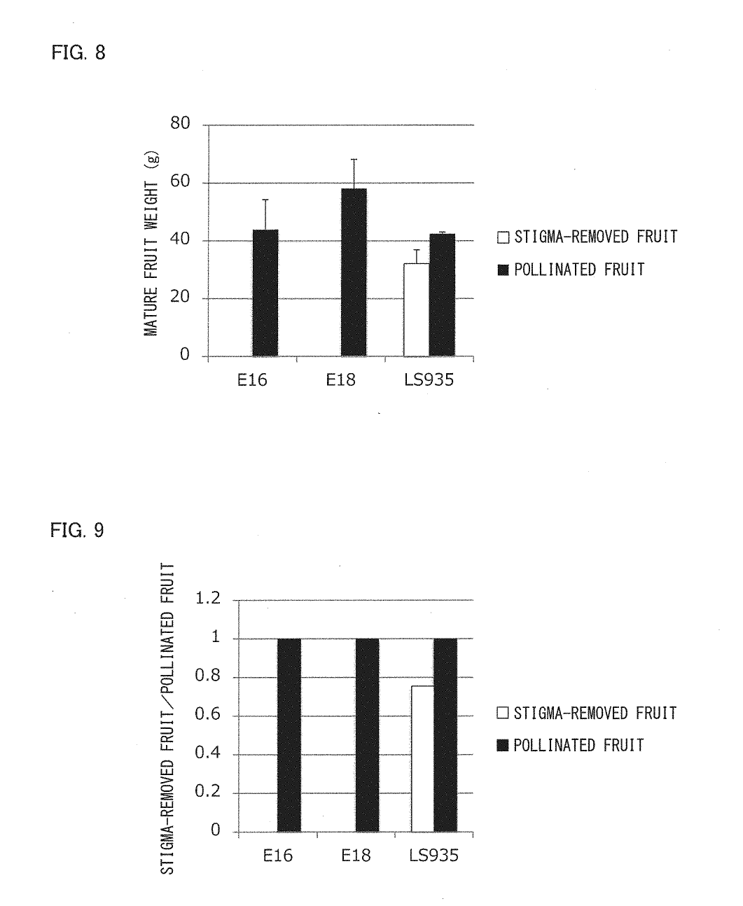

The transgenic tomatoes in each of which the parthenocarpy responsible gene A is expressed were cultivated, and mature fruit weights of stigma-removed fruits, whose stigma had been removed before anthesis, were measured and mature fruit weights of pollinated fruits, which had been pollinated at anthesis, were measured. Moreover, non-transgenic plants (LS935) were concurrently cultivated, and mature fruit weights were measured as controls through processes similar to those carried out on the transgenic tomatoes in each of which the parthenocarpy responsible gene A is expressed. Results are shown in FIGS. 8 and 9. Note that, in FIGS. 8 and 9, each of E16 and E18 is the transgenic tomato in which the parthenocarpy responsible gene A is expressed.

As shown in FIG. 8, the stigma-removed fruit of the non-transgenic tomato LS935, which is a parthenocarpic line, was fructified and enlarged to an extent similar to that of the pollinated fruit, whereas the stigma-removed fruit of the transgenic tomato in which the parthenocarpy responsible gene A is expressed was not fructified and enlarged. Moreover, as shown in FIG. 9, a ratio of the fruit weight of the stigma-removed fruit of the non-transgenic tomato LS935 was not significantly different from a ratio of the fruit weight of the pollinated fruit which was assumed to be 1. On the other hand, a ratio of the fruit weight of the stigma-removed fruit of the transgenic tomato in which the parthenocarpy responsible gene A is expressed was nearly 0.

[2. Segregation Analysis of Tomato Traits with Use of DNA Marker Sequence]

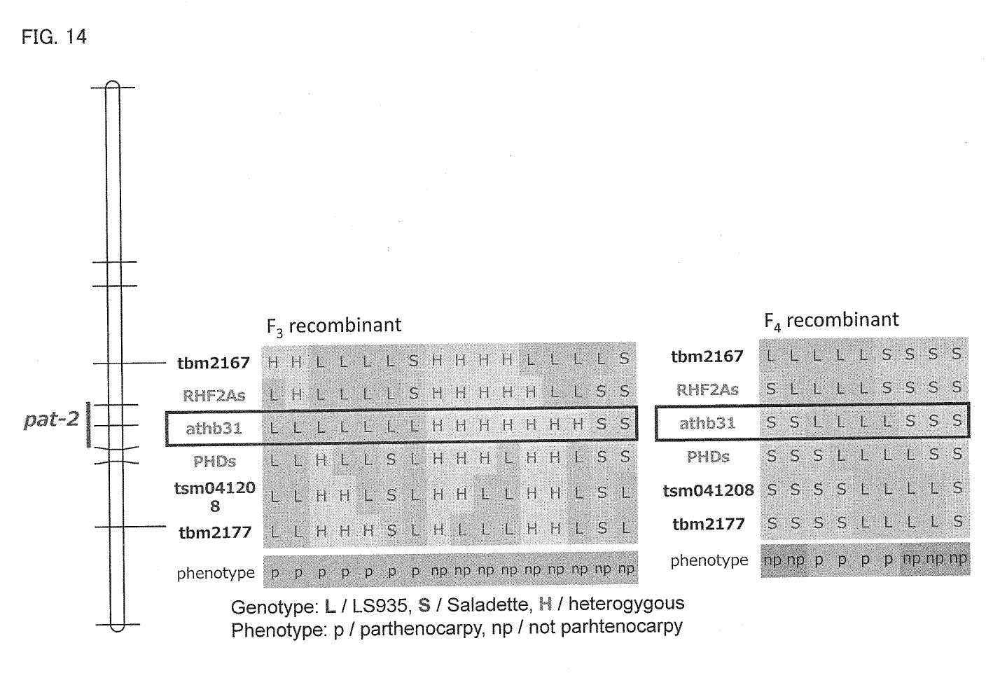

By crossing a parthenocarpic tomato line LS935 (obtained from National Institute of Vegetable and Tea Science) and a non-parthenocarpic tomato line Saladette (LA2662) (obtained from Tomato Genetics Resource Center in US), a population of F₂ individuals was obtained. Segregation of genotype and phenotype of tomato was analyzed with the use of (i) a selfed progeny F₃ population which was a line selected from the F₂ population, (ii) a selfed progeny F₄ population which was a line selected from the F₃ population, and (iii) marker sequences. Results are shown in FIG. 14.

In FIG. 14, a genotype indicated by “L” is a homozygote of LS935 (in which deletion occurs in the parthenocarpy responsible gene: parthenocarpy (p)), a genotype indicated by “S” is a homozygote of Saladette (in which no deletion occurs in the parthenocarpy responsible gene A: not parthenocarpy (np)), and a genotype indicated by “H” is a homozygote (not parthenocarpy (np)).

The marker sequences in FIG. 14 were developed by the inventors on their own and have characteristics below. Among these, RHF2As, athb31, and PHDs are high in evaluation accuracy, and particularly athb31 is a complete linkage marker that achieves evaluation accuracy of 100%.
tbm2167: has a distance of approximately 1.1 cM from the parthenocarpy responsible gene A; is a SSR marker which is amplified by PCR that is carried out with the use of (i) primers respectively represented by SEQ ID NO: 48 and SEQ ID NO: 49 located on SEQ ID NO: 47 and (ii) genomic DNA as a template; and is a parthenocarpic line LS935 type if a fragment length is 241 base pairs or is a non-parthenocarpic line Saladette type if the fragment length is 243 base pairs.
RHF2As: has a distance of approximately 135.1 kb from the parthenocarpy responsible gene A; is SNP marker whose 123th nucleotide is A or T in a base sequence represented by SEQ ID NO: 50; and has a genotype of the parthenocarpic line LS935 if the nucleotide is A or has a genotype of the non-parthenocarpic line Saladette if the nucleotide is T.
athb31: is an insertion-deletion mutation in the parthenocarpy responsible gene A; is a mutation in which 9977th through 11011th nucleotides are deleted from the base sequence represented by SEQ ID NO: 3; and has a genotype of the parthenocarpic line LS935 if the deletion has been caused or has a genotype of the non-parthenocarpic line Saladette if the deletion has not been caused.
PHDs: has a distance of approximately 161.6 kb from the parthenocarpy responsible gene A; is SNP whose 119th nucleotide is A or G in a base sequence represented by SEQ ID NO: 51; and has a genotype of the parthenocarpic line LS935 if the nucleotide is A or has a genotype of the non-parthenocarpic line Saladette if the nucleotide is G.
tsm041208: has a distance of approximately 247.7 kb from the parthenocarpy responsible gene A; is a polymorphism of a fragment which is amplified by PCR that is carried out with the use of (i) primers respectively represented by SEQ ID NO: 53 and SEQ ID NO: 54 located on SEQ ID NO: 52 and (ii) genomic DNA as a template; and is the parthenocarpic line LS935 type if a fragment length is 393 base pairs or is the non-parthenocarpic line Saladette type if the fragment length is 359 base pairs.
tbm2177: has a distance of approximately 5.3 cM from the parthenocarpy responsible gene A; is a SSR marker which is amplified by PCR that is carried out with the use of (i) primers respectively represented by SEQ ID NO: 56 and SEQ ID NO: 57 located on SEQ ID NO: 55 and (ii) genomic DNA as a template; and is the parthenocarpic line LS935 type if a fragment length is 243 base pairs or is the non-parthenocarpic line Saladette type if the fragment length is 241 base pairs.

[3. Identification of Eggplant-Derived Parthenocarpy Responsible Gene SmA]

A sequencing library was prepared from genomic DNA of an eggplant cultivar “Nakateshinkuro”, and pair end sequence of a fragment having an insert size of 200 pb to 300 pb and mate pair sequence of a fragment having an insert size of 2 kb were carried out with the use of a sequencer HiSeq2000 (manufactured by illumina Inc.) A total of 14.4 hundred-millions reads thus obtained (i.e., sequence data corresponding to approximately 140× of eggplant genome (approximately 1.1 Gb)) were assembled by an assembler program SOAPdenovo, and 1,321,157 sequences and fragment genomic sequences having a full length of 1,145 Mb was obtained. BLASTN analysis was carried out with respect to the sequence, and a genomic sequence (67,902 base pairs) was obtained which partially showed high homology to the tomato parthenocarpy responsible gene A. Gene prediction was carried out with the use of the genomic sequence thus obtained and a gene prediction program Augustus, and (i) a genomic DNA sequence (SEQ ID NO: 34) which includes a homologue gene (hereinafter, referred to as “parthenocarpy responsible gene SmA) candidate of eggplant and (ii) a protein code sequence (SEQ ID NO: 35) of the genomic DNA sequence were obtained. From the base sequences respectively represented by SEQ ID NO: 34 and SEQ ID NO: 35, it is clear that the parthenocarpy responsible gene SmA includes two exons and one intron. PCR was carried out with the use of (i) PCR primer pairs designed based on the predicted gene sequence and (ii) cDNA derived from a bud of eggplant as a template, a cDNA clone containing entire ORF of the parthenocarpy responsible gene SmA was obtained. A base sequence of the cDNA clone thus obtained was determined by the Sanger's method, and it was confirmed that (i) the base sequence of the cDNA clone was identical with the base sequence represented by SEQ ID NO: 35 predicted by the program and (ii) the cDNA clone was a gene expressed in the bud of eggplant. The protein encoded by the parthenocarpy responsible gene SmA has the amino acid sequence represented by SEQ ID NO: 25.

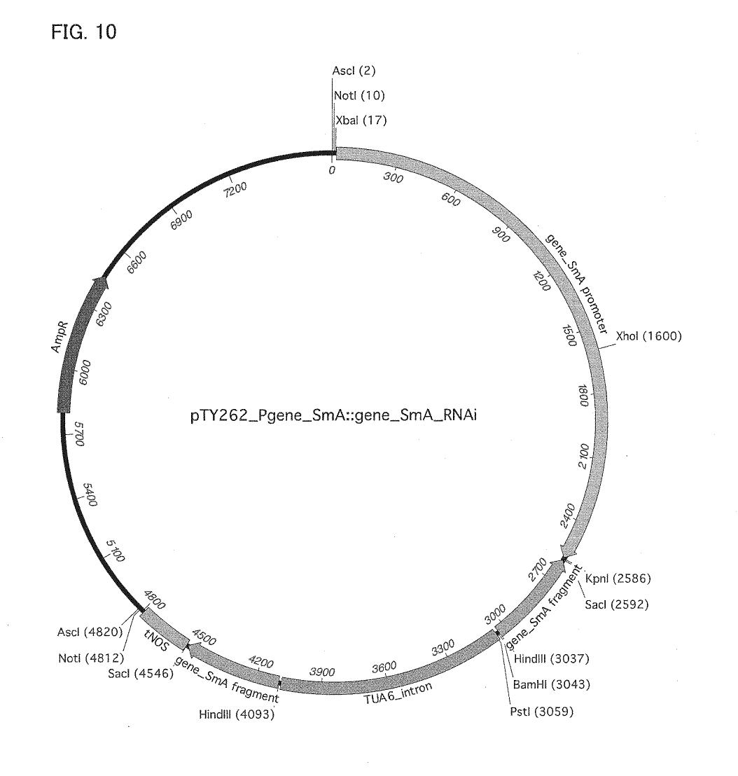

Primers having base sequences respectively represented by SEQ ID NO: 36 and SEQ ID NO: 37 were prepared, and PCR was carried out while using, as a template, a clone having a base sequence represented by SEQ ID NO: 35. A PCR product was obtained which had a base sequence of 473 base pairs represented by SEQ ID NO: 38. The PCR product had a BamHI recognition site and a HindIII recognition site at a 5′ end, and a Sad recognition site and a KpnI recognition site at a 3′ end. The PCR product was inserted to a cloning vector pTY262 (AB736152 (DDBJ)) so as to form an inverted repeat structure, and an RNAi-induction vector with respect to the parthenocarpy responsible gene SmA of eggplant was constructed. Further, a CaMV35S promoter of the vector was replaced with a SmA gene promoter sequence including 2561 base pairs represented by SEQ ID NO: 39 (see FIG. 10). The promoter sequence is predicted to include, at a 3′ end, (i) 29 base pairs which are at a 5′ end of the ORF of SmA having a base sequence represented by SEQ ID NO: 40 and (ii) a 5′ untranslated region for encoding unidentified mRNA. However, RNAi induction does not require transcription of RNA for encoding protein having a function, and therefore a function as the RNAi-induction vector is not influenced.

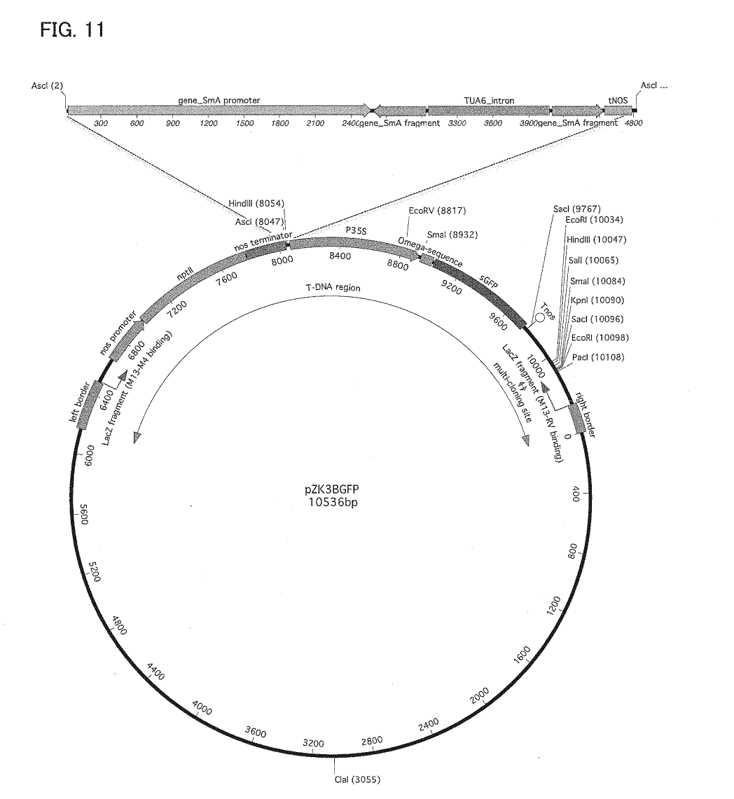

The RNAi-induction vector thus obtained was digested by AscI and then inserted into an AscI recognition site of a modified binary vector pZK3BGFP in which a reporter gene including a CaMV35S promoter, GFP, and a Nos terminator was inserted into a binary vector pZK3B (Kuroda et al., Biosci Biotech Biochem, (2010) 74: 2348-2351). Thus, an RNAi-induction binary vector pZK3BGFP_SmA-RNAi was constructed (see FIG. 11).

Transformation of a non-parthenocarpic eggplant cultivar “Senryo-nigo” was carried out by the use of the RNAi induction chimeric gene (binary vector) illustrated in FIG. 11. Specifically, with the use of the Agrobacterium method (Billings et al. 1997 J. Amer. Hort. Sci. 122: 158-162), a cotyledon of the eggplant cultivar “Senryo-nigo” was infected, and culture on a kanamycin-containing medium and selection were carried out. Then, induction of an intended gene into the regenerated plant was confirmed by PCR, and thus an RNAi transgenic eggplant was obtained.

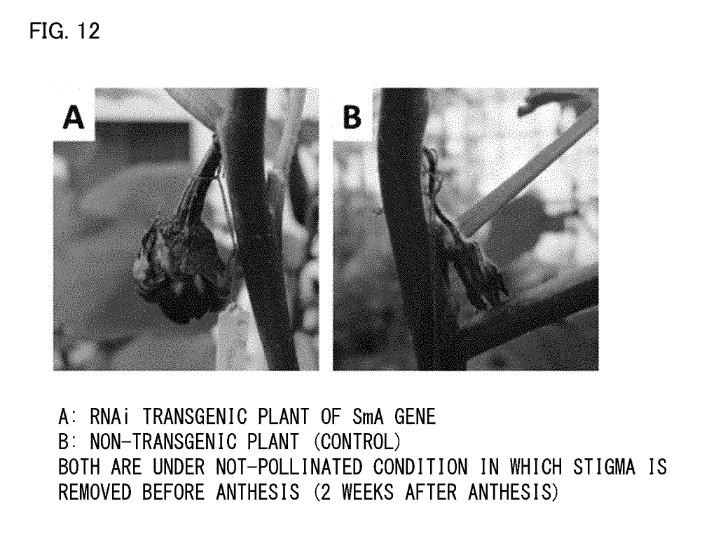

The RNAi transgenic eggplant was cultivated, and its flower whose stigma had been removed before anthesis clearly showed enlargement of an ovary (see FIG. 12). In a case where a stigma of a non-transgenic eggplant has been removed, enlargement of its ovary is hardly shown and its flower dies down in approximately 1 week after anthesis. From the above result, it has been concluded that prevention of falling and enlargement of fruit are also achieved in eggplant by inhibiting expression of the parthenocarpy responsible gene SmA, as observed in tomato.

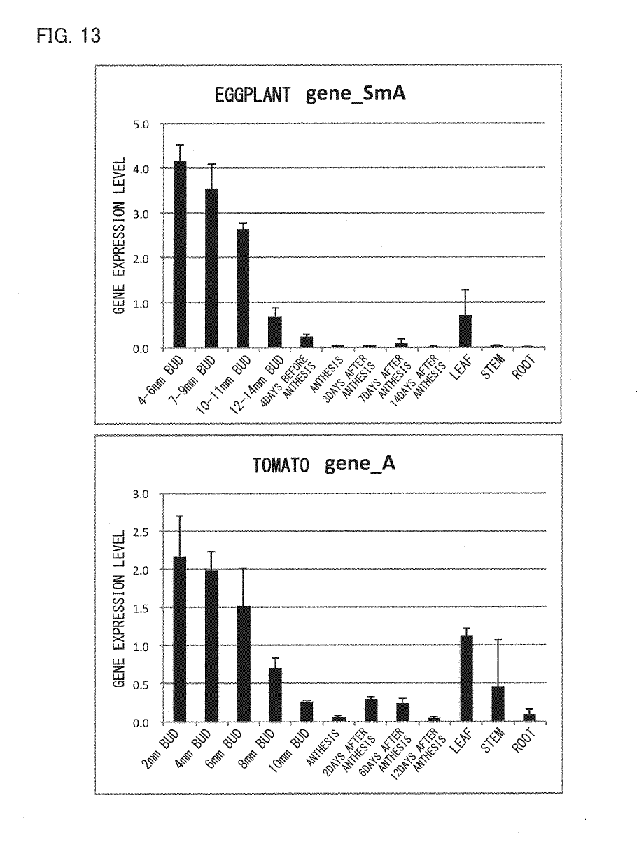

The parthenocarpy responsible gene SmA of eggplant has a sequence that is structurally most similar to that of the parthenocarpy responsible gene A of tomato. As shown in FIG. 13, it is clear that stage-specific and tissue-specific expressions are also similar to those of the parthenocarpy responsible gene A of tomato, from a result of quantitative real time PCR analysis. Note that, as an endogenous standard gene for the quantitative real time PCR, a housekeeping gene SmFL20F10 (SEQ ID NO: 41) was used which had been developed by the inventors and constitutively expressed. Moreover, for tomato, Solyc04g049180.2.1 (ITAG2.30, SEQ ID NO: 42) was used which was strongly supposed to be an orthologue of the SmFL20F10 from a result of BLAST search. The followings are sequences of the primers used to amplify the endogenous standard gene:

<For eggplant SmFL20F10 (SEQ ID NO: 41)>

Forward primer: SEQ ID NO: 43

Reverse primer: SEQ ID NO: 44

<For tomato Solyc04g049180.2.1 (SEQ ID NO: 42)>

Forward primer: SEQ ID NO: 45

Reverse primer: SEQ ID NO: 46

### INDUSTRIAL APPLICABILITY

The present invention makes it possible to obtain a new cultivar plant having parthenocarpy. Moreover, the present invention can be used in a field such as agriculture or horticulture.

1. A method for evaluating parthenocarpy of a plant, said method comprising the step of:
checking whether or not gene expression of a parthenocarpy regulatory gene, which includes a polynucleotide recited in any of (1) through (4) below, is inhibited in the plant; or
checking whether or not a function of a polypeptide, which is encoded by the parthenocarpy regulatory gene, is inhibited in the plant
(1) A polynucleotide that encodes a polypeptide having an amino acid sequence represented by SEQ ID NO: 1,
(2) a polynucleotide that encodes a polypeptide (i) having a sequence identity of 70% or higher relative to the amino acid sequence represented by SEQ ID NO: 1 and (ii) having a parthenocarpy regulation activity,
(3) a polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and
4) a polynucleotide that (i) is hybridized with a polynucleotide, which has a sequence complementary to the polynucleotide recited in the above (1), under a stringent condition and (ii) encodes a polypeptide having a parthenocarpy regulation activity.

2. The method as set forth in claim 1, wherein:
in the step of checking, a base sequence of the parthenocarpy regulatory gene or an expression level of the parthenocarpy regulatory gene is checked.

3. The method as set forth in claim 1, wherein:
in the step of checking, a plant in which the gene expression of the parthenocarpy regulatory gene is inhibited or a plant in which the function of the polypeptide encoded by the parthenocarpy regulatory gene is inhibited is selected as a parthenocarpic plant.

4. A method for producing a parthenocarpy-regulated plant, said method comprising the step of:
selecting a parthenocarpic plant by carrying out a method recited in claim 1.

5. A method for producing a parthenocarpy-regulated plant, said method comprising the step of:
inhibiting, in a plant, gene expression of a parthenocarpy regulatory gene that includes a polynucleotide recited in any of (1) through (4) below; or
inhibiting, in a plant, a function of a polypeptide encoded by the parthenocarpy regulatory gene.
(1) A polynucleotide that encodes a polypeptide having an amino acid sequence represented by SEQ ID NO: 1,
(2) a polynucleotide that encodes a polypeptide (i) having a sequence identity of 70% or higher relative to the amino acid sequence represented by SEQ ID NO: 1 and (ii) having a parthenocarpy regulation activity,
(3) a polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and
(4) a polynucleotide that (i) is hybridized with a polynucleotide, which has a sequence complementary to the polynucleotide recited in the above (1), under a stringent condition and (ii) encodes a polypeptide having a parthenocarpy regulation activity.

6. The method as set forth in claim 5, wherein:
in the step, (i) a polynucleotide, which inhibits the gene expression of the parthenocarpy regulatory gene, is introduced into the plant or (ii) the parthenocarpy regulatory gene is disrupted.

7. The method as set forth in claim 1, wherein the plant is a solanaceous plant.

8. The method as set forth in claim 7, wherein the plant is a tomato.

9. A parthenocarpy-regulated plant produced by a method recited in claim 4.

10. A parthenocarpy regulatory gene that includes a polynucleotide recited in any of (1) through (4) below.
(1) A polynucleotide that encodes a polypeptide having an amino acid sequence represented by SEQ ID NO: 1,
(2) a polynucleotide that encodes a polypeptide (i) having a sequence identity of 70% or higher relative to the amino acid sequence represented by SEQ ID NO: 1 and (ii) having a parthenocarpy regulation activity,
(3) a polynucleotide that encodes a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and
(4) a polynucleotide that (i) is hybridized with a polynucleotide, which has a sequence complementary to the polynucleotide recited in the above (1), under a stringent condition and (ii) encodes a polypeptide having a parthenocarpy regulation activity.

11. A polypeptide recited in any of (1) through (4) below.
(1) A polypeptide having an amino acid sequence represented by SEQ ID NO: 1,
(2) a polypeptide (i) having a sequence identity of 70% or higher relative to the amino acid sequence represented by SEQ ID NO: 1 and (ii) having a parthenocarpy regulation activity,
(3) a polypeptide (i) having an amino acid sequence in which 1 to 87 amino acids are substituted in, deleted from, inserted into, and/or added to the amino acid sequence represented by SEQ ID NO: 1 and (ii) having a parthenocarpy regulation activity, and
(4) a polypeptide that (i) is encoded by a polynucleotide which is hybridized, under a stringent condition, with a polynucleotide that has a sequence complementary to a polynucleotide for encoding the polypeptide recited in the above (1) and (ii) has a parthenocarpy regulation activity.

12. The method as set forth in claim 5, wherein the plant is a solanaceous plant.

13. The method as set forth in claim 12, wherein the plant is a tomato.

14. A parthenocarpy-regulated plant produced by a method recited in claim 5.


SEQUENCE LISTING (57 sequences)

SEQ ID NO: 1 - protein, 290 aa, Solanum lycopersicum
MALGGGNEEKEMRISGFQNIDLVSPGGAAPDDIVSAAPHNNSTTGSNKLNKGTSTVRGRYRECLKNHAANIGGNVTDGCGEFMPSGEEGTLEALKCAACNCHRNFHRKEIPNNVGAGVGGGVGGDNNNNAGIMVVHPLQLPQPLPSPIPSLNHHHHHHHQHGGRSIWTTMPPQPVKMAFGGSGGGSGATDSSSEELNFNTYHHQQATSVPPQQPFMLAKKRFRTKFSQEQKEKMLEFAEKLGWRIPREDDAEVQRFCSQVGVKRQVFKVWMHNNKNPSSAKKNIIQEDQP

SEQ ID NO: 2 - DNA, 873 bp, Solanum lycopersicum
atggcattaggtggtggtaatgaggaaaaagagatgagaatttcaggttttcaaaatatagatcttgtctcacctggtggtgcagcgccagacgacatcgtcagcgctgcaccacacaataattccactactggatctaataaattgaacaagggaactagtacagtgagaggtagatatcgtgagtgcttgaaaaatcatgctgctaatattggtggaaatgttacggatggttgtggtgagtttatgcctagtggtgaagaaggtacactcgaagctttgaaatgtgctgcttgtaattgtcataggaattttcatagaaaagaaatacctaacaacgttggcgctggtgttggtggtggtgttggtggtgataataataataatgcggggattatggttgtgcatccattacagctgccacaaccactaccttctccgataccgtcgttgaatcatcatcaccaccatcaccatcaacacggaggaagatcaatttggactactatgccacctcagcctgtcaaaatggccttcggaggtagcggcggaggaagtggggcaacagactcatcaagtgaagaacttaatttcaacacatatcatcatcaacaagcaacatcagtacctccacaacaaccatttatgttggctaagaaaagatttaggacaaaatttagtcaagaacagaaggaaaaaatgttggaatttgctgaaaaattaggttggagaattccaagagaagatgatgctgaagtacaaagattttgctcacaagttggagtcaagagacaggtttttaaggtttggatgcataataataagaacccttcttctgctaaaaaaaatataatacaagaagatcaaccttaa

SEQ ID NO: 3 - DNA, 13050 bp, Solanum lycopersicum
cctgaccgtggtgttcaggtcaacttctgctcaactagttcgacgagatacctgtcacgtcccaccagcaataggtatcaggtaactctgtcgaccaaggttaggacaactcttcatgctgaagaaactacaactcgtgcatctggagtctagacaaatagatggttgatactagtttcgaaaagagctttagagtcaaagacaagtcactttcattagttgtaacaaataacaagactgcagttatagtataggagtacaagagaaaataattacataaaaaacatataagttagctagtgaagaggctacctatatatgttttacgccatgataaacctcatggtacctacaccttatcttgaaaagacaaaagcaggggaaattgtgaaagacgcggtattagaaataagcatagggttagatttttttcgatacaactataggcaacctaatagttaaaaccatccaaatgtaagtcgataaaaacttaaaggaaacagacgaaattgagataatgcataacattaagtacttagtctatgcagtttgttcacaattcgttatcatcacaacatacttgaagtcaatatacagattgtcaaaaagattgtttttttggcctaaaatactatagcgttgacgcgtctatattagccatagaattggaattggaggatctaatctcttccagcagggtgaatttcctatctgatattatccacttgctgatgataatacttttcaacaaaaagtttggtcacaaaatactattttcttattctccctccacctattgtacctctccttaaagaaaaatattcgacaacttgctacaactcaacgccttcttctccatcttataaggacctaacgtagcattatgttgcccataccattcacttgagctagcactcggctttaggcgaagtagctctgcacacacgtgtacaacatggcctcgtggactgcaaggcaaaacaagaagttcgaagaggctttggcgttatatgacagagacacacctgatcgttggcataacattgcaaggtgtgtaggtggaaaatcagctgcagaagtgaagaggcattatgaagtacttgtgaaggatatcatgcaaatagaaaatggtcaagtacctttaccaaattacaaagccgcggctgaaaccaataacagatcaggttatgccaatgaacataggtattaaacatcaaaactaatcttttattaattttgtacaagtcattgatacgttctgtattcgattcccatatggttcgtgtcattttcctattcatggtactgtttatgtttatcaccatctatccatttttagtaactttacaagccaaagactagtcacactccttgttgacacttttattctaaagaataaatgcttatagtttcaactctctgcatccacgattgtttcttgttcctcgataaaagaaaatgtacgcaaacaagtaactaatgcttatgaacatgtctatatgttgttgaacaggcttctgaagaacctgaagttacagtgagaacaaatttcgcggccatgctgaataacatcacaagttgtctgttatatttgcaaatgcaaaaggcataaattgtgaatttatgtcttaagtttttgaagcaagcaaaatgtattatgactattgttgttcatctctatccatttaacttttgttttactgttgtaatgtttctcaaacgcataatataaagtctacttcactttcatgttatagtgtaatatgtttgataaaataaaatcaaaattacacgttttatagtctgaattcaatggatcgtaattctcaattcatttgttcttttttagatacttgtaaaatattcaagtacactttcacaatgagattgtcacacggttcagtatcataacaatcaaatctcacaccgtacatgagttcatatgtgattctttaggttactattagagattacgaattcgagctatgggagctaacaacatcatgagttcatatgtgattctttaggttattattagagattacgaattcgagctaggagctaacatctaaaaaatagttcatatgtgattctttaggttattattagagattacgaattcgagctaggagctaacatctaaaaaataaaagtaggaacgtaaaaatggagatgcgggtatcgatccccgtacctctcgcatgctaagcgagcgctctaccatctgagctacatcccctttcacatgctgtgcatcttatattaaaataaatatatcaaataaacatctagtaaacagaaatagttgtacaaggaaggaacatttagcactttttttttttaagaatcaaaactgcttaaatgacatatttatgtattataccattataaaactaaatcaattcagactcgctttaaaaaattgtatacaacgtctatatattaatttttatctcataggatcaatcacaaaattaattttgtaatacatcaacaaggtagattaaaattttacaggaacacctttacatgtagaaaattattattgccattgaataccaagaacaaaaaaaaatatactattttctcttgtacatagttaggtaactaacaaactaatcaaactcccctcagcttttttcgagcttctgtctgtcaaaggttcgagaatcaatgaaagatcatcaccgtctttgttataggcgagcaaattaacagacgaaaataaaaaaggaaaaatcttgaacacccaacacatctcctcttcgtcttcttcgtatgaccaaatttgtaccaattcttgagtctcataactataagcaacaattttatgtgccaaaacttgtagatataatacctgtaaatccttattgaatgcacatggcttcactgtgtaccctgtttcaaaccccaatcttacaaaatgatcaagcttgaagtcatttcctctgaaactccatatgaaaactccattgtcaactggatcacagaattgtattactccgtcttcttccgatgattcgtataaaaactgagagtggtaaacatcttttcttctggaattgtttttcggcagtgaaaacagtgtaaatactttgaaatctatgtcgtataccaacaaacataaactccatatccagtacaattttcttttctcgcgagaaaaaacactcgaattatagcattgccctcgaataaatccatctgattggaggagatgggaattcaaaatttgaatttgggtttcccgccacttccatgaattcgatccgaagataatacaattcaccatttgggtatcgaaatcaatttcttcccagaagaaagcgataacttgaaacgaatttaactcaaaaacaagacttgaacaagtgggtcttgaaatatgatgagaaattggtagagaaattaactggttaagaacaggggatgaaacagagtatgtccagaacccattgttgttattttctgaagcgtaaagaagaaccccattacaagaatcgatcaaataagggaatttatgagtggaagatgaatgggaagtacaaaatttggggatttttttctgggttaagtcgtgtaacttcatgaaaactgattcttgataagttttgtgtttgaggaagatcacaagaccagttgaagttgtgagagggtaacaatggcggatattggtaatcgaatttcgccatgttttcgaaacaatcttcaattggatgagagatttaaagggaattttagtgataattgattcaactatatgatttgataaattcccaagctctatcatctcttcttcttcttcttgctctgtttccatggcaatcgccattgctaatcccctctgttttttgaagttaaatttcatattcaatcaattaattaatacttaaaaggtatttatagaaaccctaataaactagctatggcggttaattatcaattacagttaatttatttgtgtttgataattaattaattgattaattgtacttggtatgcaagattataagtaagtaaatgcttgatgagcaagcattaaatttctatagttaaacgggttgataactaatcacctatattttctcacttaaatatttatttttggctatttatttacaatttttttggctagttatatttttggtaaagtttaatcatgtaatatatgacatatgtatatatataatttatagcgtaactttcacatataacaaataaaaaatttatatttgcatgctatagtaaagtttgcgtaattgcgctccatagcaaacataaaaattgtataattcgctatacatatacagttgaagcaaattgtataaaacgaagtgtataaaacaagaaagagaaagacacttgggcagagaactgtataaaaacgaagtgtataaaacatattgtattataagtgtatagaacgattatatacaatttgaatttgtataaaatgagaatgaaagaaaacaaaagagacttgacaaggaatatacaattgaatcgaattgtataaaacgagaaatggagaaattagatacaatttaaaaattgtataaaacgagaaagagtgaaaggcaaaaaaaaacgggatagggaaatatttttattgtataatcataagtgtataggacaaatagcaaaactatagagcgctaatatgattttatatttattattcctaaaatttactattctaaaaattattttaaaatactcataagtttgtattatcattctataatgaaaagtgtttcgtgaaaagtcattacaagaacataattcaaattcattaaaatgaatttgaaaaaaaaggaaggaaataaaagaaaaatgtcattgatttgtgtatgaaaaactactattattacttgtaattggaagtgacttcgaagaacaagctaggataaaaatagtatataaagtatttatataaataaataaattttattcaacaaattaataatgaaatttaatcaaaaattttgaatgataggtgtttttattcagctactagtaatagtgtgattatttaagagtgtatttatatattattttatgcatgtattaattatatagagattgattatttattcactcccataaggtatctatttcttatttcatgcataaaaagatacataaattctttcataacttttacatattttaatagtcatgcaagtttcttaaatatatacatgaattagatatttttctatacctatcgaacataaatgtcgtacaaattatataaaaattaatataaaaataacttatctcttatcagctatcaaacaacttgtaatttcacaaataaaatatgaaaatgttaataggtaaactataaatatgttatatttaatagaatctaacactaataaacaattcaaagtaaagaaaaaaaattgctataacgttgctctcaattaaatcagatgtcataattttgacgtttcacaaaaatagaattttttttaaagaaaattgtcatatttgttgaagtaacatgtgtgcccatatgtggtcctgttattattagaaagtaggtaaaagaagtatgaaaaatactatattataattggatctcatgatataataagtgaacataatttggcacagattaagaaaagcatcctcaattaaggatattaatagaatatttattcatgccataaaaggaaagaatagtataattgcatgtgtttttttaattacatgtttgcaagactcaaattgttatgtttattagtcggtgcttaccttaaaataataaatgcatatattatttgcacatcaactttttctatttaattatagttgactaacatgtacttttatattctaataaagtctacctaattatgataagacaattctccctcattaacttttgtttgttttatgttatataaattttagtgattcaaatcttaattattagtgtatatttgttatattactttataaccatgcacttaacgattttaaatagatttgaataatttataatatttttagttattcagaaaaatacacaagtatctttttaatctatgttcaaaaaacttcaaagacacacttatactatactaatgttctattacttccctaaatttattttatagtaattttctacctctttttgatctacgtggtattgggttgaaaaaaaaataaaccagttttgatcccacaaaatagtgtcacgtagaccaaaagggggaaggaaaattacatataaaataagttcaggggaataatatgaccttggtatagtataaatgtgtctttagattttggcataggttgagaaagtatttgagcattatccctatttataatcatttacaatcatcattacccataataatcgttcatcataattattagttatcacttctggcttcaactaatcacaaaacttttaaaaaatcattaatatgtcatataaaaattatttataaaaagtaataattttaccgttactttgatatttcatcaaaaacatacaaaaaaacttttaatggatgcatttcccaagttatcatataaattgataatgaccacaagtagccttctgattcctcctatggtccattatcttcatgtttcaacatttatagcttctgcttcttgcatagcttatttaaataaaattacattttaatatatatatagaaaaagtgaaacaaactttcatagacccaatttaataatataccatttgtttccattaattttaaaagtgaatggaaattaaccacactcacaaacaaatatgatgaaataacatattttattcatcactatacctaaatacaatcatatttttcacttatttttatttatgaattttcttcgcgacttgatgattttaatatctaatggatgtatgttgatttgaatatccttgcaatgtgttttattattacgtatgtcatgtattgttatttaatatgttgattttttaataaaaaaaatatttataaaaaattattcgagaatataataatactagaaatatttaaaataatcgcttctttcaataaaacaatatatcttgctacatgtacacctttgagtttcacttgcttattatattgtttatttttggcgatttatttttttatatatattattgaacatgatccaaagaagatccaccattgttcgtttccaagcttatttattaatttatttattttcctctattttaatttgacggtgaaatatgattatcaattctttcttccattttaataatcaaaattacttaaaatatccaaaatgattgtaattttacaaaaaataaaatgaaattaaataatattaagttttcactttgcctttaatattaagtttttacggaagcaaccattatactaaacaataggaagaaaacaatataaatccgaacaaataaataattatcggttgatatcatgattttcaaatataaaaaagattaattcatattcagtcatatttcttaaaaataaataaggacacaaagaaaaacatgagaattattaataatatagaatgaatgaagcatgttgaatttatatattatataaaatatattttttaattagtctcaattaatatttatattttcaattttaaaaattcataaataaaaaattaaatttacataaaataaaataaattaatacatatatttaacactatataaaacaaatatatgtcacgtaaattttgtttactgttactttattgacgtatattaaatgttagtaataggataaaaactttcaatcattgcaaggaaaagatggtgtatgcattgtatttagcaattaattttatgaataatattaatgcctaatttatgtatttttatatttttttaagtgacaataaattaatgtgacgcaattatgctgtctcaattgtcgagcccctgcaatattactgataacgcaaatgccttgaatgttgattaatctgtaaaaaaagataaagcaagtaccaaagcaaataacagaaattagacatgacaagagacctcacccagatgtaagtttgtactaattttattttttatcttaaattatttatcatatcttgataagttgattagcgtagacacttaaatttgtataaagttgaacaaataggcacacttgaccattttatatggtaatttatatcttacgtgtactatgtcacatatggctcatgtgtctacctgtttaattttatataagtttaagtttttacttgtgtgcattcaaaattgagatgcataattgtgtaactaatttaaattaaatgagacatttaccatgtcttgtgtcatttaaaaataaaaaatacttactattagagaaaatctctctcgttaaaatttgaatggtgacaaataaattgaaacgagaaaaaagaatttgctaaggagaaaagcatggagtttgcaggtagaaggtgacatagaattaggagtactcataatttgcatcattcattgggcagaagttgaataataataataaaagctgatagcagagtcaaatcagattgcagaatcgcactatttttgcgtcattttcgactctatgtgtatgtaacacggtaaactaaaagattcaccaactccacaccccaaaacatatggtgaatcttttgaagactagagtagggatatcgccaaatatgtcctaagaagctgaggactaagtttaatatatccataatttaaatttatagatggagtactagataattttatattaaactaatttttatcaaatccaacctaattcattttttaagtagtattcaaactttgcccatttctaagtcatacttatatttacttttgactttttttccctatagtttggagcttaagttctgagtcagagttaaaacttgaaattaaagttggattgctcactggttaaggttggatctcaggtcgagatcggtttattgttcaggactcgagacaaagtcatctcatgccccaaatccctgtcgggtatcgagtcccaagtaagaattgaatgtcgaatcacgagtcttgaatttcaaattaagcccgaagctcaattatcgagtctaaaatctcgagtcagaggcgaggtcgagtctagagtcctggtcagggttagatttcaagtaaggatcaaaattgagactcaaaatctcaaattttataactccattcaaaatcaaatcaagtctgagatccggactcaaggatcgaattagagaaataaagttgttcattctaaccccctaatgggaaaatgaaggggggggggttggtagggcaataatgaataaatggggtggtgggcatggggccaatgtttgcagggcgtgggttaggactgaacatgacattgtcggttgttttcttatgcacacttcatccagtatctctttgctgtttcccactttctctctcttctctctctctctcccttcttctttagctccgtttgaagcaaccatgttgtctcttgtttcttacatcccatgtttcttcctccccaacaacaccactttttattccttaaaatctctccttcttttttaacaacatagtccattgtttttccttctctacttctagggccccccatcctcctttcatttcttcaatttcacacccccccacataacattagacatagggttagggtttctttttttttttttttaaattaacaaaaaaaaaaaaatcatggcattaggtggtggtaatgaggaaaaagagatgagaatttcaggttttcaaaatatagatcttgtctcacctggtggtgcagcgccagacgacatcgtcagcgcgcaccacacaataattccactactggatctaataaattgaacaagggaactagtacagtgagaggtagatatcgtgagtgcttgaaaaatcatgctgctaatattggtggaaatgttacggatggttgtggtgagtttatgcctagtggtgaagaaggtacactcgaagctttgaaatgtgctgcttgtaattgtcataggaattttcatagaaaagaaatacctaacaacgttggcgctggtgttggtggtggtgttggtggtgataataataataatgcggggattatggttgtgcatccattacagctgccacaaccactaccttctccgataccgtcgttgaatcatcatcaccaccatcaccatcaacacggaggaagatcaatttggactactatgccacctcagcctgtcaaaatggccttcggaggtaatgtgtttagctcatctaaaaattttaatgaaatctcatttgctcaatttgaattaccatgccacctcagcctatcaaaatgggctttggggggtttcgatttaacattcgattatttcgtctaatagtttaaattgtgagacatattttttttatttacttgtactaattatttcattttatgtgtctttgtttcacattgcactaaatttagaaggtgaatatctaatttgaatattgtgattttaaaccttattaatcatttcttaaaatagtatcattaagaataattagcatcatgagaatgttagatttcaaagcttacttaaatactcatatgataagatataaacatacattactagaaataaataaaaaaaattcaccttgaattcgtgagaaatttgtattatcgaactgatttttccactcatttttcacctaaccagctcacggccgcgagaaacacatctaacttcctaattttttcgcgagaaaatgttctggtcattccttactaattcaatcaaaatatctctaaatatttctccatgaaagaaataataattttttagtaatgatatttactgaagacacaaattaaaataaattattacgcttaattatgtcttcaacacgttgttagtacctttttgtcgactcgaacctaagactcttgatatcatgatgaatcgtatgataagttaaaatcattaaagtgaactatatacctacatttttatttttttatttacttaattatatcttcaaacaggtagcggcggaggaagtggggcaacagactcatcaagtgaagaacttaatttcaacacatatcatcatcaacaagcaacatcagtacctccacaacaaccatttatgttggctaagaaaagatttaggacaaaatttagtcaagaacagaaggaaaaaatgttggaatttgctgaaaaattaggttggagaattccaagagaagatgatgctgaagtacaaagattttgctcacaagttggagtcaagagacaggtttttaaggtttggatgcataataataagaaccctttttttgctaaaaaaaatataatacaagaagatcaaccttaattaattttgtttaattaaggttttgagggaaaaaatgaaattaaatttcttgggatttggtttatgcttatgtaatcttactaggaatcgtacgtagctagttttttttgttttttttttgggggggtctatgtagtattattgtaactatatggaaccacttaattagatttttatttttattaattgttgtactttggaacctttattcatctgaaaattttcagctatttcttacacttctcttgtcactgaaatttagaccactcatgatgccgctttatcgaggttagcgaggtagagataaaatttacgtatgtaaactttaaaaaaaatgtgctagatatgttgttgtaatatgagccaagttgtatatcgaattctttagctctcaagatagagtaaagtctgtgtatgaagttagttaattgatgatgttgagggagaaacatgagttatttatttttttgaaatataaaaatgacaagtaaaagcgaaaaataaatgttttttttaggaatggaaggatatttaattaatagagagccgttggatgaaaagaaagaatgaatcttagtcgtctaatttgaattatttgaaagagtagtagttttttttatccttagtcttgtcgaatataaaagcttttcctatttaaatttatttattagtttgtgtattgtctatttcaataaattatgatgcttcttggagtttctgtcaccattttaaatgtttgtttcctattttgggaatagtttgctgcttctttttctcactatttgaggcttatacttatagagcatatcaccccaatcttctccatgtgattatcaatgttgagtgtcataaacattagataatacaaattttcgacattcgatttaagattatgattttaaaattatttttagatatgtaattttattatcatgattttaaagtttaaattattcaagaagcacgattgaaattttagttgatttatattttataaaaaaaatatatcattgaattttgaaaaaagaaagacccatgctcatcgtgaattgacagtttatgctatgtgaaataattatactaagaaatttctctttatattccttttagttgttctgaaattctttttctcaaagttaaaaaataagaattttgattaacgttttacaatacattttattattatattgataataataagaactttaactaattttattagatgttgcgatcagatacatagccctacgcgacttatcgagacgttgagtgatgtatctgaaactgatcagatgcaatatattgagacgtaatgtattgagatattttggaaagcaattgaattccactaaattaaaaaaaagtatatatataataaaagagagtaagaatataatatatttaactctttaacacgtatgcaatgttatgttaaattcccttaataattcacttaaaaggttatttgaactttgattaagatgttgtgaataatagatcatagttcaagtccattcatttcttatcaaattttaatcatcctcatttttgtctctttttcatgtgctttgacgtttgattgcttctatccttatcttcacttatttgatataaaaaaacaaaaaatacattaaaaatgtaggtcactaaattctacataaaacacattattcaaagtaata

SEQ ID NO: 4 - DNA, 12033 bp, Solanum lycopersicum
cctgaccgtggtgttcaggtcaacttctgctcaactagttcgacgagatacctgtcacgtcccaccagcaataggtatcaggtaactctgtcgaccaaggttaggacaactcttcatgctgaagaaactacaactcgtgcatctggagtctagacaaatagatggttgatactagtttcgaaaagagctttagagtcaaagacaagtcactttcattagttgtaacaaataacaagactgcagttatagtataggagtacaagagaaaataattacataaaaaacatataagttagctagtgaagaggctacctatatatgttttacgccatgataaacctcatggtacctacaccttatcttgaaaagacaaaagcaggggaaattgtgaaagacgcggtattagaaataagcatagggttagatttttttcgatacaactataggcaacctaatagttaaaaccatccaaatgtaagtcgataaaaacttaaaggaaacagacgaaattgagataatgcataacattaagtacttagtctatgcagtttgttcacaattcgttatcatcacaacatacttgaagtcaatatacagattgtcaaaaagattgtttttttggcctaaaatactatagcgttgacgcgtctatattagccatagaattggaattggaggatctaatctcttccagcagggtgaatttcctatctgatattatccacttgctgatgataatacttttcaacaaaaagtttggtcacaaaatactattttcttattctccctccacctattgtacctctccttaaagaaaaatattcgacaacttgctacaactcaacgccttcttctccatcttataaggacctaacgtagcattatgttgcccataccattcacttgagctagcactcggctttaggcgaagtagctctgcacacacgtgtacaacatggcctcgtggactgcaaggcaaaacaagaagttcgaagaggctttggcgttatatgacagagacacacctgatcgttggcataacattgcaaggtgtgtaggtggaaaatcagctgcagaagtgaagaggcattatgaagtacttgtgaaggatatcatgcaaatagaaaatggtcaagtacctttaccaaattacaaagccgcggctgaaaccaataacagatcaggttatgccaatgaacataggtattaaacatcaaaactaatcttttattaattttgtacaagtcattgatacgttctgtattcgattcccatatggttcgtgtcattttcctattcatggtactgtttatgtttatcaccatctatccatttttagtaactttacaagccaaagactagtcacactccttgttgacacttttattctaaagaataaatgcttatagtttcaactctctgcatccacgattgtttcttgttcctcgataaaagaaaatgtacgcaaacaagtaactaatgcttatgaacatgtctatatgttgttgaacaggcttctgaagaacctgaagttacagtgagaacaaatttcgcggccatgctgaataacatcacaagttgtctgttatatttgcaaatgcaaaaggcataaattgtgaatttatgtcttaagtttttgaagcaagcaaaatgtattatgactattgttgttcatctctatccatttaacttttgttttactgttgtaatgtttctcaaacgcataatataaagtctacttcactttcatgttatagtgtaatatgtttgataaaataaaatcaaaattacacgttttatagtctgaattcaatggatcgtaattctcaattcatttgttcttttttagatacttgtaaaatattcaagtacactttcacaatgagattgtcacacggttcagtatcataacaatcaaatctcacaccgtacatgagttcatatgtgattctttaggttactattagagattacgaattcgagctatgggagctaacaacatcatgagttcatatgtgattctttaggttattattagagattacgaattcgagctaggagctaacatctaaaaaatagttcatatgtgattctttaggttattattagagattacgaattcgagctaggagctaacatctaaaaaataaaagtaggaacgtaaaaatggagatgcgggtatcgatccccgtacctctcgcatgctaagcgagcgctctaccatctgagctacatcccctttcacatgctgtgcatcttatattaaaataaatatatcaaataaacatctagtaaacagaaatagttgtacaaggaaggaacatttagcactttttttttttaagaatcaaaactgcttaaatgacatatttatgtattataccattataaaactaaatcaattcagactcgctttaaaaaattgtatacaacgtctatatattaatttttatctcataggatcaatcacaaaattaattttgtaatacatcaacaaggtagattaaaattttacaggaacacctttacatgtagaaaattattattgccattgaataccaagaacaaaaaaaaatatactattttctcttgtacatagttaggtaactaacaaactaatcaaactcccctcagcttttttcgagcttctgtctgtcaaaggttcgagaatcaatgaaagatcatcaccgtctttgttataggcgagcaaattaacagacgaaaataaaaaaggaaaaatcttgaacacccaacacatctcctcttcgtcttcttcgtatgaccaaatttgtaccaattcttgagtctcataactataagcaacaattttatgtgccaaaacttgtagatataatacctgtaaatccttattgaatgcacatggcttcactgtgtaccctgtttcaaaccccaatcttacaaaatgatcaagcttgaagtcatttcctctgaaactccatatgaaaactccattgtcaactggatcacagaattgtattactccgtcttcttccgatgattcgtataaaaactgagagtggtaaacatcttttcttctggaattgtttttcggcagtgaaaacagtgtaaatactttgaaatctatgtcgtataccaacaaacataaactccatatccagtacaattttcttttctcgcgagaaaaaacactcgaattatagcattgccctcgaataaatccatctgattggaggagatgggaattcaaaatttgaatttgggtttcccgccacttccatgaattcgatccgaagataatacaattcaccatttgggtatcgaaatcaatttcttcccagaagaaagcgataacttgaaacgaatttaactcaaaaacaagacttgaacaagtgggtcttgaaatatgatgagaaattggtagagaaattaactggttaagaacaggggatgaaacagagtatgtccagaacccattgttgttattttctgaagcgtaaagaagaaccccattacaagaatcgatcaaataagggaatttatgagtggaagatgaatgggaagtacaaaatttggggatttttttctgggttaagtcgtgtaacttcatgaaaactgattcttgataagttttgtgtttgaggaagatcacaagaccagttgaagttgtgagagggtaacaatggcggatattggtaatcgaatttcgccatgttttcgaaacaatcttcaattggatgagagatttaaagggaattttagtgataattgattcaactatatgatttgataaattcccaagctctatcatctcttcttcttcttcttgctctgtttccatggcaatcgccattgctaatcccctctgttttttgaagttaaatttcatattcaatcaattaattaatacttaaaaggtatttatagaaaccctaataaactagctatggcggttaattatcaattacagttaatttatttgtgtttgataattaattaattgattaattgtacttggtatgcaagattataagtaagtaaatgcttgatgagcaagcattaaatttctatagttaaacgggttgataactaatcacctatattttctcacttaaatatttatttttggctatttatttacaatttttttggctagttatatttttggtaaagtttaatcatgtaatatatgacatatgtatatatataatttatagcgtaactttcacatataacaaataaaaaatttatatttgcatgctatagtaaagtttgcgtaattgcgctccatagcaaacataaaaattgtataattcgctatacatatacagttgaagcaaattgtataaaacgaagtgtataaaacaagaaagagaaagacacttgggcagagaactgtataaaaacgaagtgtataaaacatattgtattataagtgtatagaacgattatatacaatttgaatttgtataaaatgagaatgaaagaaaacaaaagagacttgacaaggaatatacaattgaatcgaattgtataaaacgagaaatggagaaattagatacaatttaaaaattgtataaaacgagaaagagtgaaaggcaaaaaaaaacgggatagggaaatatttttattgtataatcataagtgtataggacaaatagcaaaactatagagcgctaatatgattttatatttattattcctaaaatttactattctaaaaattattttaaaatactcataagtttgtattatcattctataatgaaaagtgtttcgtgaaaagtcattacaagaacataattcaaattcattaaaatgaatttgaaaaaaaaggaaggaaataaaagaaaaatgtcattgatttgtgtatgaaaaactactattattacttgtaattggaagtgacttcgaagaacaagctaggataaaaatagtatataaagtatttatataaataaataaattttattcaacaaattaataatgaaatttaatcaaaaattttgaatgataggtgtttttattcagctactagtaatagtgtgattatttaagagtgtatttatatattattttatgcatgtattaattatatagagattgattatttattcactcccataaggtatctatttcttatttcatgcataaaaagatacataaattctttcataacttttacatattttaatagtcatgcaagtttcttaaatatatacatgaattagatatttttctatacctatcgaacataaatgtcgtacaaattatataaaaattaatataaaaataacttatctcttatcagctatcaaacaacttgtaatttcacaaataaaatatgaaaatgttaataggtaaactataaatatgttatatttaatagaatctaacactaataaacaattcaaagtaaagaaaaaaaattgctataacgttgctctcaattaaatcagatgtcataattttgacgtttcacaaaaatagaattttttttaaagaaaattgtcatatttgttgaagtaacatgtgtgcccatatgtggtcctgttattattagaaagtaggtaaaagaagtatgaaaaatactatattataattggatctcatgatataataagtgaacataatttggcacagattaagaaaagcatcctcaattaaggatattaatagaatatttattcatgccataaaaggaaagaatagtataattgcatgtgtttttttaattacatgtttgcaagactcaaattgttatgtttattagtcggtgcttaccttaaaataataaatgcatatattatttgcacatcaactttttctatttaattatagttgactaacatgtacttttatattctaataaagtctacctaattatgataagacaattctccctcattaacttttgtttgttttatgttatataaattttagtgattcaaatcttaattattagtgtatatttgttatattactttataaccatgcacttaacgattttaaatagatttgaataatttataatatttttagttattcagaaaaatacacaagtatctttttaatctatgttcaaaaaacttcaaagacacacttatactatactaatgttctattacttccctaaatttattttatagtaattttctacctctttttgatctacgtggtattgggttgaaaaaaaaataaaccagttttgatcccacaaaatagtgtcacgtagaccaaaagggggaaggaaaattacatataaaataagttcaggggaataatatgaccttggtatagtataaatgtgtctttagattttggcataggttgagaaagtatttgagcattatccctatttataatcatttacaatcatcattacccataataatcgttcatcataattattagttatcacttctggcttcaactaatcacaaaacttttaaaaaatcattaatatgtcatataaaaattatttataaaaagtaataattttaccgttactttgatatttcatcaaaaacatacaaaaaaacttttaatggatgcatttcccaagttatcatataaattgataatgaccacaagtagccttctgattcctcctatggtccattatcttcatgtttcaacatttatagcttctgcttcttgcatagcttatttaaataaaattacattttaatatatatatagaaaaagtgaaacaaactttcatagacccaatttaataatataccatttgtttccattaattttaaaagtgaatggaaattaaccacactcacaaacaaatatgatgaaataacatattttattcatcactatacctaaatacaatcatatttttcacttatttttatttatgaattttcttcgcgacttgatgattttaatatctaatggatgtatgttgatttgaatatccttgcaatgtgttttattattacgtatgtcatgtattgttatttaatatgttgattttttaataaaaaaaatatttataaaaaattattcgagaatataataatactagaaatatttaaaataatcgcttctttcaataaaacaatatatcttgctacatgtacacctttgagtttcacttgcttattatattgtttatttttggcgatttatttttttatatatattattgaacatgatccaaagaagatccaccattgttcgtttccaagcttatttattaatttatttattttcctctattttaatttgacggtgaaatatgattatcaattctttcttccattttaataatcaaaattacttaaaatatccaaaatgattgtaattttacaaaaaataaaatgaaattaaataatattaagttttcactttgcctttaatattaagtttttacggaagcaaccattatactaaacaataggaagaaaacaatataaatccgaacaaataaataattatcggttgatatcatgattttcaaatataaaaaagattaattcatattcagtcatatttcttaaaaataaataaggacacaaagaaaaacatgagaattattaataatatagaatgaatgaagcatgttgaatttatatattatataaaatatattttttaattagtctcaattaatatttatattttcaattttaaaaattcataaataaaaaattaaatttacataaaataaaataaattaatacatatatttaacactatataaaacaaatatatgtcacgtaaattttgtttactgttactttattgacgtatattaaatgttagtaataggataaaaactttcaatcattgcaaggaaaagatggtgtatgcattgtatttagcaattaattttatgaataatattaatgcctaatttatgtatttttatatttttttaagtgacaataaattaatgtgacgcaattatgctgtctcaattgtcgagcccctgcaatattactgataacgcaaatgccttgaatgttgattaatctgtaaaaaaagataaagcaagtaccaaagcaaataacagaaattagacatgacaagagacctcacccagatgtaagtttgtactaattttattttttatcttaaattatttatcatatcttgataagttgattagcgtagacacttaaatttgtataaagttgaacaaataggcacacttgaccattttatatggtaatttatatcttacgtgtactatgtcacatatggctcatgtgtctacctgtttaattttatataagtttaagtttttacttgtgtgcattcaaaattgagatgcataattgtgtaactaatttaaattaaatgagacatttaccatgtcttgtgtcatttaaaaataaaaaatacttactattagagaaaatctctctcgttaaaatttgaatggtgacaaataaattgaaacgagaaaaaagaatttgctaaggagaaaagcatggagtttgcaggtagaaggtgacatagaattaggagtactcataatttgcatcattcattgggcagaagttgaataataataataaaagctgatagcagagtcaaatcagattgcagaatcgcactatttttgcgtcattttcgactctatgtgtatgtaacacggtaaactaaaagattcaccaactccacaccccaaaacatatggtgaatcttttgaagactagagtagggatatcgccaaatatgtcctaagaagctgaggactaagtttaatatatccataatttaaatttatagatggagtactagataattttatattaaactaatttttatcaaatccaacctaattcattttttaagtagtattcaaactttgcccatttctaagtcatacttatatttacttttgactttttttccctatagtttggagcttaagttctgagtcagagttaaaacttgaaattaaagttggattgctcactggttaaggttggatctcaggtcgagatcggtttattgttcaggactcgagacaaagtcatctcatgccccaaatccctgtcgggtatcgagtcccaagtaagaattgaatgtcgaatcacgagtcttgaatttcaaattaagcccgaagctcaattatcgagtctaaaatctcgagtcagaggcgaggtcgagtctagagtcctggtcagggttagatttcaagtaaggatcaaaattgagactcaaaatctcaaattttataactccattcaaaatcaaatcaagtctgagatccggactcaaggatcgaattagagaaataaagttgttcattctaaccccctaatgggaaaatgaaggggggggggttggtagggcaataatgaataaatggggtggtgggcatggggccaatgtttgcagggcgtgggttaggactgaacatgacattgtcggttgttttcttatgcacacttcatccagtatctctttgctgtttcccactttctctctcttctctctctctctcccttcttctttagctccgtttgaagcaaccatgttgtctcttgtttcttacatcccatgtttcttcctccccaacaacaccactttttattccttaaaatctctccttcttttttaacaacatagtccattgtttttccttctctacttctagggccccccatcctcctttcatttcttcaatttcacacccccccacataacattagacatagggttagggtttcttttttttttttttaaattaaaaaaaaaaaaaaaatcttttcatttggtggtgtaaaggaggaaaaaaagatgtgtttttcaggttatcaaaatatcgctcttgtctccccgggggtggccgcgccgggaggacttggtgagggctccaccacacaataattccactactggatctaataaattgaacaagggaactagtacagtgagaggtagatatcgtgagtgcttgaaaaatcatgctgctaatattggtggaaatgttacggatggttgtggtgagtttatgcctagtggtgagtttacttaattatatcttcaaacaggtagcggcggaggaagtggggcaacagactcatcaagtgaagaacttaatttcaacacatatcatcatcaacaagcaacatcagtacctccacaacaaccatttatgttggctaagaaaagatttaggacaaaatttagtcaagaacagaaggaaaaaatgttggaatttgctgaaaaattaggttggagaattccaagagaagatgatgctgaagtacaaagattttgctcacaagttggagtcaagagacaggtttttaaggtttggatgcataataataagaaccctttttttgctaaaaaaaatataatacaagaagatcaaccttaattaattttgtttaattaaggttttgagggaaaaaatgaaattaaatttcttgggatttggtttatgcttatgtaatcttactaggaatcgtacgtagctagttttttttgttttttttttgggggggtctatgtagtattattgtaactatatggaaccacttaattagatttttatttttattaattgttgtactttggaacctttattcatctgaaaattttcagctatttcttacacttctcttgtcactgaaatttagaccactcatgatgccgctttatcgaggttagcgaggtagagataaaatttacgtatgtaaactttaaaaaaaatgtgctagatatgttgttgtaatatgagccaagttgtatatcgaattctttagctctcaagatagagtaaagtctgtgtatgaagttagttaattgatgatgttgagggagaaacatgagttatttatttttttgaaatataaaaatgacaagtaaaagcgaaaaataaatgttttttttaggaatggaaggatatttaattaatagagagccgttggatgaaaagaaagaatgaatcttagtcgtctaatttgaattatttgaaagagtagtagttttttttatccttagtcttgtcgaatataaaagcttttcctatttaaatttatttattagtttgtgtattgtctatttcaataaattatgatgcttcttggagtttctgtcaccattttaaatgtttgtttcctattttgggaatagtttgctgcttctttttctcactatttgaggcttatacttatagagcatatcaccccaatcttctccatgtgattatcaatgttgagtgtcataaacattagataatacaaattttcgacattcgatttaagattatgattttaaaattatttttagatatgtaattttattatcatgattttaaagtttaaattattcaagaagcacgattgaaattttagttgatttatattttataaaaaaaatatatcattgaattttgaaaaaagaaagacccatgctcatcgtgaattgacagtttatgctatgtgaaataattatactaagaaatttctctttatattccttttagttgttctgaaattctttttctcaaagttaaaaaataagaattttgattaacgttttacaatacattttattattatattgataataataagaactttaactaattttattagatgttgcgatcagatacatagccctacgcgacttatcgagacgttgagtgatgtatctgaaactgatcagatgcaatatattgagacgtaatgtattgagatattttggaaagcaattgaattccactaaattaaaaaaaagtatatatataataaaagagagtaagaatataatatatttaactctttaacacgtatgcaatgttatgttaaattcccttaataattcacttaaaaggttatttgaactttgattaagatgttgtgaataatagatcatagttcaagtccattcatttcttatcaaattttaatcatcctcatttttgtctctttttcatgtgctttgacgtttgattgcttctatccttatcttcacttatttgatataaaaaaacaaaaaatacattaaaaatgtaggtcactaaattctacataaaacacatattcaaagtaatactacaaaaaactaaag

SEQ ID NO: 5 - DNA, 1034 bp, Solanum lycopersicum
agaaggtacactcgaagctttgaaatgtgctgcttgtaattgtcataggaattttcatagaaaagaaatacctaacaacgttggcgctggtgttggtggtggtgttggtggtgataataataataatgcggggattatggttgtgcatccattacagctgccacaaccactaccttctccgataccgtcgttgaatcatcatcaccaccatcaccatcaacacggaggaagatcaatttggactactatgccacctcagcctgtcaaaatggccttcggaggtaatgtgtttagctcatctaaaaattttaatgaaatctcatttgctcaatttgaattaccatgccacctcagcctatcaaaatgggctttggggggtttcgatttaacattcgattatttcgtctaatagtttaaattgtgagacatattttttttatttacttgtactaattatttcattttatgtgtctttgtttcacattgcactaaatttagaaggtgaatatctaatttgaatattgtgattttaaaccttattaatcatttcttaaaatagtatcattaagaataattagcatcatgagaatgttagatttcaaagcttacttaaatactcatatgataagatataaacatacattactagaaataaataaaaaaaattcaccttgaattcgtgagaaatttgtattatcgaactgatttttccactcatttttcacctaaccagctcacggccgcgagaaacacatctaacttcctaattttttcgcgagaaaatgttctggtcattccttactaattcaatcaaaatatctctaaatatttctccatgaaagaaataataattttttagtaatgatatttactgaagacacaaattaaaataaattattacgcttaattatgtcttcaacacgttgttagtacctttttgtcgactcgaacctaagactcttgatatcatgatgaatcgtatgataagttaaaatcattaaagtgaactatatacctacatttttattttttta

SEQ ID NO: 6 - DNA, 873 bp, Solanum lycopersicum; identical to SEQ ID NO: 2

SEQ ID NO: 7 - DNA, 23 bp, Artificial Sequence
  primer sequence
tgttgccccacttcctccgccgc

SEQ ID NO: 8 - DNA, 23 bp, Artificial Sequence
  primer sequence
cggcggaggaagtggggcaacag

SEQ ID NO: 9 - DNA, 20 bp, Artificial Sequence
  primer sequence
gctttgaaatgtgctgcttg

SEQ ID NO: 10 - DNA, 22 bp, Artificial Sequence
  primer sequence
ttcagcatcatcttctcttgga

SEQ ID NO: 11 - DNA, 1405 bp, Solanum lycopersicum
gtttcccactttctctctcttctctctctctctcccttcttctttagctccgtttgaagcaaccatgttgtctcttgtttcttacatcccatgtttcttcctccccaacaacaccactttttattccttaaaatctctccttcttttttaacaacatagtccattgtttttccttctctacttctagggccccccatcctcctttcatttcttcaatttcacacccccccacataacattagacatagggttagggtttctttttttttttttttaaattaacaaaaaaaaaaaaatcatggcattaggtggtggtaatgaggaaaaagagatgagaatttcaggttttcaaaatatagatcttgtctcacctggtggtgcagcgccagacgacatcgtcagcgctgcaccacacaataattccactactggatctaataaattgaacaagggaactagtacagtgagaggtagatatcgtgagtgcttgaaaaatcatgctgctaatattggtggaaatgttacggatggttgtggtgagtttatgcctagtggtgaagaaggtacactcgaagctttgaaatgtgctgcttgtaattgtcataggaattttcatagaaaagaaatacctaacaacgttggcgctggtgttggtggtggtgttggtggtgataataataataatgcggggattatggttgtgcatccattacagctgccacaaccactaccttctccgataccgtcgttgaatcatcatcaccaccatcaccatcaacacggaggaagatcaatttggactactatgccacctcagcctgtcaaaatggccttcggaggtagcggcggaggaagtggggcaacagactcatcaagtgaagaacttaatttcaacacatatcatcatcaacaagcaacatcagtacctccacaacaaccatttatgttggctaagaaaagatttaggacaaaatttagtcaagaacagaaggaaaaaatgttggaatttgctgaaaaattaggttggagaattccaagagaagatgatgctgaagtacaaagattttgctcacaagttggagtcaagagacaggtttttaaggtttggatgcataataataagaacccttcttctgctaaaaaaaatataatacaagaagatcaaccttaattaattttgtttaattaaggttttgagggaaaaaatgaaattaaatttcttgggatttggtttatgcttatgtaatcttactaggaatcgtacgtagctagttttttttgttttttttttgggggggtctatgtagtattattgtaactatatggaaccacttaattagatttttatttttattaattgttgtactttggaacctttattcatctgaaaattttcagctatttc

SEQ ID NO: 12 - DNA, 2572 bp, Solanum lycopersicum
aagcttatttattaatttatttattttcctctattttaatttgacggtgaaatatgattatcaattctttcttccattttaataatcaaaattacttaaaatatccaaaatgattgtaattttacaaaaaataaaatgaaattaaataatattaagttttcactttgcctttaatattaagtttttacggaagcaaccattatactaaacaataggaagaaaacaatataaatccgaacaaataaataattatcggttgatatcatgattttcaaatataaaaaagattaattcatattcagtcatatttcttaaaaataaataaggacacaaagaaaaacatgagaattattaataatatagaatgaatgaagcatgttgaatttatatattatataaaatatattttttaattagtctcaattaatatttatattttcaattttaaaaattcataaataaaaaattaaatttacataaaataaaataaattaatacatatatttaacactatataaaacaaatatatgtcacgtaaattttgtttactgttactttattgacgtatattaaatgttagtaataggataaaaactttcaatcattgcaaggaaaagatggtgtatgcattgtatttagcaattaattttatgaataatattaatgcctaatttatgtatttttatatttttttaagtgacaataaattaatgtgacgcaattatgctgtctcaattgtcgagcccctgcaatattactgataacgcaaatgccttgaatgttgattaatctgtaaaaaaagataaagcaagtaccaaagcaaataacagaaattagacatgacaagagacctcacccagatgtaagtttgtactaattttattttttatcttaaattatttatcatatcttgataagttgattagcgtagacacttaaatttgtataaagttgaacaaataggcacacttgaccattttatatggtaatttatatcttacgtgtactatgtcacatatggctcatgtgtctacctgtttaattttatataagtttaagtttttacttgtgtgcattcaaaattgagatgcataattgtgtaactaatttaaattaaatgagacatttaccatgtcttgtgtcatttaaaaataaaaaatacttactattagagaaaatctctctcgttaaaatttgaatggtgacaaataaattgaaacgagaaaaaagaatttgctaaggagaaaagcatggagtttgcaggtagaaggtgacatagaattaggagtactcataatttgcatcattcattgggcagaagttgaataataataataaaagctgatagcagagtcaaatcagattgcagaatcgcactatttttgcgtcattttcgactctatgtgtatgtaacacggtaaactaaaagattcaccaactccacaccccaaaacatatggtgaatcttttgaagactagagtagggatatcgccaaatatgtcctaagaagctgaggactaagtttaatatatccataatttaaatttatagatggagtactagataattttatattaaactaatttttatcaaatccaacctaattcattttttaagtagtattcaaactttgcccatttctaagtcatacttatatttacttttgactttttttccctatagtttggagcttaagttctgagtcagagttaaaacttgaaattaaagttggattgctcactggttaaggttggatctcaggtcgagatcggtttattgttcaggactcgagacaaagtcatctcatgccccaaatccctgtcgggtatcgagtcccaagtaagaattgaatgtcgaatcacgagtcttgaatttcaaattaagcccgaagctcaattatcgagtctaaaatctcgagtcagaggcgaggtcgagtctagagtcctggtcagggttagatttcaagtaaggatcaaaattgagactcaaaatctcaaattttataactccattcaaaatcaaatcaagtctgagatccggactcaaggatcgaattagagaaataaagttgttcattctaaccccctaatgggaaaatgaaggggggggggttggtagggcaataatgaataaatggggtggtgggcatggggccaatgtttgcagggcgtgggttaggactgaacatgacattgtcggttgttttcttatgcacacttcatccagtatctctttgctgtttcccactttctctctcttctctctctctctcccttcttctttagctccgtttgaagcaaccatgttgtctcttgtttcttacatcccatgtttcttcctccccaacaacaccactttttattccttaaaatctctccttcttttttaacaacatagtccattgtttttccttctctacttctagggccccccatcctcctttcatttcttcaatttcacacccccccacataacattagacatagggttagggtttctttttttttttttttaaattaacaaaaaaaaaaaaatcatggcattaggtggtggtaatgaggaaaacccggg

SEQ ID NO: 13 - DNA, 31 bp, Artificial Sequence
  primer sequence
ctccccgggaagctttgaaatgtgctgcttg

SEQ ID NO: 14 - DNA, 31 bp, Artificial Sequence
  primer sequence
ctcctcgagttcagcatcatcttctcttgga

SEQ ID NO: 15 - DNA, 500 bp, Solanum lycopersicum
ctccccgggaagctttgaaatgtgctgcttgtaattgtcataggaattttcatagaaaagaaatacctaacaacgttggcgctggtgttggtggtggtgttggtggtgataataataataatgcggggattatggttgtgcatccattacagctgccacaaccactaccttctccgataccgtcgttgaatcatcatcaccaccatcaccatcaacacggaggaagatcaatttggactactatgccacctcagcctgtcaaaatggccttcggaggtagcggcggaggaagtggggcaacagactcatcaagtgaagaacttaatttcaacacatatcatcatcaacaagcaacatcagtacctccacaacaaccatttatgttggctaagaaaagatttaggacaaaatttagtcaagaacagaaggaaaaaatgttggaatttgctgaaaaattaggttggagaattccaagagaagatgatgctgaactcgaggag

SEQ ID NO: 16 - DNA, 1651 bp, Solanum lycopersicum
atggcattaggtggtggtaatgaggaaaaagagatgagaatttcaggttttcaaaatatagatcttgtctcacctggtggtgcagcgccagacgacatcgtcagcgctgcaccacacaataattccactactggatctaataaattgaacaagggaactagtacagtgagaggtagatatcgtgagtgcttgaaaaatcatgctgctaatattggtggaaatgttacggatggttgtggtgagtttatgcctagtggtgaagaaggtacactcgaagctttgaaatgtgctgcttgtaattgtcataggaattttcatagaaaagaaatacctaacaacgttggcgctggtgttggtggtggtgttggtggtgataataataataatgcggggattatggttgtgcatccattacagctgccacaaccactaccttctccgataccgtcgttgaatcatcatcaccaccatcaccatcaacacggaggaagatcaatttggactactatgccacctcagcctgtcaaaatggccttcggaggtaatgtgtttagctcatctaaaaattttaatgaaatctcatttgctcaatttgaattaccatgccacctcagcctatcaaaatgggctttggggggtttcgatttaacattcgattatttcgtctaatagtttaaattgtgagacatattttttttatttacttgtactaattatttcattttatgtgtctttgtttcacattgcactaaatttagaaggtgaatatctaatttgaatattgtgattttaaaccttattaatcatttcttaaaatagtatcattaagaataattagcatcatgagaatgttagatttcaaagcttacttaaatactcatatgataagatataaacatacattactagaaataaataaaaaaaattcaccttgaattcgtgagaaatttgtattatcgaactgatttttccactcatttttcacctaaccagctcacggccgcgagaaacacatctaacttcctaattttttcgcgagaaaatgttctggtcattccttactaattcaatcaaaatatctctaaatatttctccatgaaagaaataataattttttagtaatgatatttactgaagacacaaattaaaataaattattacgcttaattatgtcttcaacacgttgttagtacctttttgtcgactcgaacctaagactcttgatatcatgatgaatcgtatgataagttaaaatcattaaagtgaactatatacctacatttttatttttttatttacttaattatatcttcaaacaggtagcggcggaggaagtggggcaacagactcatcaagtgaagaacttaatttcaacacatatcatcatcaacaagcaacatcagtacctccacaacaaccatttatgttggctaagaaaagatttaggacaaaatttagtcaagaacagaaggaaaaaatgttggaatttgctgaaaaattaggttggagaattccaagagaagatgatgctgaagtacaaagattttgctcacaagttggagtcaagagacaggtttttaaggtttggatgcataataataagaacccttcttctgctaaaaaaaatataatacaagaagatcaaccttaa

SEQ ID NO: 17 - DNA, 617 bp, Solanum lycopersicum
atggcattaggtggtggtaatgaggaaaaagagatgagaatttcaggttttcaaaatatagatcttgtctcacctggtggtgcagcgccagacgacatcgtcagcgctgcaccacacaataattccactactggatctaataaattgaacaagggaactagtacagtgagaggtagatatcgtgagtgcttgaaaaatcatgctgctaatattggtggaaatgttacggatggttgtggtgagtttatgcctagtggtgatttacttaattatatcttcaaacaggtagcggcggaggaagtggggcaacagactcatcaagtgaagaacttaatttcaacacatatcatcatcaacaagcaacatcagtacctccacaacaaccatttatgttggctaagaaaagatttaggacaaaatttagtcaagaacagaaggaaaaaatgttggaatttgctgaaaaattaggttggagaattccaagagaagatgatgctgaagtacaaagattttgctcacaagttggagtcaagagacaggtttttaaggtttggatgcataataataagaacccttcttctgctaaaaaaaatataatacaagaagatcaaccttaa

SEQ ID NO: 18 - DNA, 717 bp, Solanum pimpinellifolium
ccacacaataattccactactggatctaataaattgaacaagggaactagtacagtgagaggtagatatcgtgagtgcttgaaaaatcatgctgctaatattggtggaaatgttacggatggttgtggtgagtttatgcctagtggtgaagaaggtacactcgaagctttgaaatgtgctgcttgtaattgtcataggaattttcatagaaaagaaatacctaacaacgttggcgttggcattggtgttggtggtgataataataataatgcggggattatggttgtgcatccattacagctgccacaaccactaccttctccgataccgtcgttgaatcatcatcaccaccatcaccatcaacacggaggaagatcaatttggactactatgccacctcagcctgtcaaaatggccttcggaggtagcggcggaggaagtggggcaacagactcatcaagtgaagaacttaatttcaacacatatcatcatcaacaagcaacatcagtacctccacaacaaccatttatgttggctaagaaaagatttaggacaaaatttagtcaagaacagaaggaaaaaatgttggaatttgctgaaaaattaggttggagaattccaagagaagatgatgctgaagtacaaagattttgctcacaagttggagtcaagagacagaccactcatgatgccgctttatcgaggttagcgaggtag

SEQ ID NO: 19 - protein, 238 aa, Solanum pimpinellifolium
PHNNSTTGSNKLNKGTSTVRGRYRECLKNHAANIGGNVTDGCGEFMPSGEEGTLEALKCAACNCHRNFHRKEIPNNVGVGIGVGGDNNNNAGIMVVHPLQLPQPLPSPIPSLNHHHHHHHQHGGRSIWTTMPPQPVKMAFGGSGGGSGATDSSSEELNFNTYHHQQATSVPPQQPFMLAKKRFRTKFSQEQKEKMLEFAEKLGWRIPREDDAEVQRFCSQVGVKRQTTHDAALSRLAR

SEQ ID NO: 20 - DNA, 1014 bp, Solanum peruvianum
aaaaaagaaaaaaaaaatagctacgtacgattcctagtaagattacataagcataaaccaaatcccaagaaatttaatttcattttttcccctcaaaaccttaattaaacaaaattaattaaggttgatcttcttgtattatattttttttagcagaagaagggttcttattattatgcatccaaaccttaaaaacctgtctcttgactccaacttgtgagcaaaatctttgtacttcagcatcatcttctcttggaattctccaacctaatttttcagcaaattccaacattttttccttttgttcttgactaaattttgtcctaaatcttttcttagccaacataaatggttgttgtggaggtactgatgttgcttgttgatgatgatatgtgttgaaattaagttcttcacttgatgagtctgttgccccacttcctccgccgctacctccgaaggccattttgacaggctgaggtggcatagtagtccaaattgatcttcctccgtgttgatggtgatggtggtggtgatgattcaacgatggtatcggagaaggtagtggttgtggtagttgtaatggatgcacaaccataatccccgcattattattattatcaccaccaacaccaacaccaccaccaacgccaacaacgttgttaggtatttcttttctatgaaaattcctatgacaattacaagcagcacatttcaaagcttcgagtgtaccttcttcaccactaggcataaactcaccacatccatccgtaacatttccaccaatattagcagcatgatttttcaagcactcacgatatctacctctcactgtactagttgccttgttcaatttattagatccagtagtggaattattgtgtggtgcagcgccaacgacgacgtcgtctggcgctgcaccaccaccaggtgagacaagatctatattttgaaaacctgaaattctcatctctttttcctcattaccaccacctaatgccatgatttttatttttt

SEQ ID NO: 21 - protein, 282 aa, Solanum peruvianum
MRISGFQNIDLVSPGGGAAPDDVVVGAAPHNNSTTGSNKLNKATSTVRGRYRECLKNHAANIGGNVTDGCGEFMPSGEEGTLEALKCAACNCHRNFHRKEIPNNVVGVGGGVGVGGDNNNNAGIMVVHPLQLPQPLPSPIPSLNHHHHHHHQHGGRSIWTTMPPQPVKMAFGGSGGGSGATDSSSEELNFNTYHHQQATSVPPQQPFMLAKKRFRTKFSQEQKEKMLEFAEKLGWRIPREDDAEVQRFCSQVGVKRQVFKVWMHNNKNPSSAKKNIIQEDQP

SEQ ID NO: 22 - DNA, 855 bp, Solanum tuberosum phureja
atggaattagctggtggtgaggaaaaggagatgagaatgtcaggttatcattcacttgatcaaaatatagatcttctctcacccaccggcggtggaggtggaggtggtggtgcagcgccaggtgatggcgctgcaccacacactaattccaccgctggatctaataaatttaacaagggaactactacagcaagagcaaggtaccgtgagtgtctgaaaaatcatgctgctaatattggtggaaatgttactgatggatgtggtgagtttatgcctagtggtggagaaggtacacttgaagctttgaaatgtgctgcttgtaattgtcatcggaattttcatagaaaagaacagcctaataataatgccgggattatggttgtgcatccattacagctaccacaatcactgccttctccgataccatcgttgaatcaccaccaccaccatcaccatcaacacggaggaggaagatcaatttggactactatgccacctcagcctgtcaaaatggcctttggaggtagcgggggaggaagtggggcaacagactcatcaagtgaagaacttaatttcaatacatatcatcatcaacaagcaacatcggtacatccacaacaaccatttatgttggctaagaaaagatttaggacaaaatttagtcaagaacaaaaggaaaaaatgttggaatttgctgaaaaattaggttggagaattccaagagaagatgatactgaagtacaaagattctgctcacaagttggagtcaagagacaggtttttaaggtttggatgcataataataagaatccttcttctgctaaaaaaaacataatacaagaagatcaaccttaa

SEQ ID NO: 23 - protein, 284 aa, Solanum tuberosum phureja
MELAGGEEKEMRMSGYHSLDQNIDLLSPTGGGGGGGGAAPGDGAAPHTNSTAGSNKFNKGTTTARARYRECLKNHAANIGGNVTDGCGEFMPSGGEGTLEALKCAACNCHRNFHRKEQPNNNAGIMVVHPLQLPQSLPSPIPSLNHHHHHHHQHGGGRSIWTTMPPQPVKMAFGGSGGGSGATDSSSEELNFNTYHHQQATSVHPQQPFMLAKKRFRTKFSQEQKEKMLEFAEKLGWRIPREDDTEVQRFCSQVGVKRQVFKVWMHNNKNPSSAKKNIIQEDQP

SEQ ID NO: 24 - DNA, 879 bp, Solanum melongena
atggcattaaccggtgaggaaaaggaaatgagaatgtcaggttccttaggatatcattcacttgatcaaaacatagatcacactgtcccaccaaacattcaagatcaatctaccggcggaggtagtggtgcagcgccagttgctggcgttgcaccacataattcctccgccggatctaataaattcaataaggtaacttcatcaagagctaggtaccgtgagtgtctgaaaaatcatgctgctaacattggtggaaatgttaccgatggatgtggtgaattcatgcctagtggagaagaaggaactcttgaggctttgaaatgtgctgcgtgtaattgtcaccgtaattttcacagaaaagaactgcccaacaatggtgataataataataacaacccggggattatggttgtccatccactgcaacttcctcaaccacttcctactccaataccgtcgatgaatcaccatcatcaccatcaccaccaacacagaggtcgatcaatctggactaccatgccacctcagcctgtcaaaatggccttcggagccagcgggggaagtggggcaacagactcatcaagtgaagaacttaatttcaacacttatcatcatcaacaggcaacctcagttcctccacaacaaccatttttgttggctaagaaaaggtttaggacaaaattcagtcaagaacaaaaggaaaaaatgttggaatttgcagagaaattaggatggagaattccaagagaagatgatactgaagtacaaagtttctgctcacaagttggagtgaagagacaggtatttaaggtttggatgcataataataagaacccttctgctaaaaagaacataaatcaagaagatcaacaaccttaa

SEQ ID NO: 25 - protein, 292 aa, Solanum melongena
MALTGEEKEMRMSGSLGYHSLDQNIDHTVPPNIQDQSTGGGSGAAPVAGVAPHNSSAGSNKFNKVTSSRARYRECLKNHAANIGGNVTDGCGEFMPSGEEGTLEALKCAACNCHRNFHRKELPNNGDNNNNNPGIMVVHPLQLPQPLPTPIPSMNHHHHHHHQHRGRSIWTTMPPQPVKMAFGASGGSGATDSSSEELNFNTYHHQQATSVPPQQPFLLAKKRFRTKFSQEQKEKMLEFAEKLGWRIPREDDTEVQSFCSQVGVKRQVFKVWMHNNKNPSAKKNINQEDQQP

SEQ ID NO: 26 - DNA, 20 bp, Artificial Sequence
  primer sequence
ggggattatggttgtgcatc

SEQ ID NO: 27 - DNA, 20 bp, Artificial Sequence
  primer sequence
gatgagtctgttgccccact

SEQ ID NO: 28 - DNA, 729 bp, Solanum lycopersicum
atggggtatgcaatcatcagcactattggacttcttttcttggggaagctgcttgagcccatctggggttctagggaattcttaaagttcatctttattgtcaactttttgacatctgtctttgttttcatcacagccatatcattgtactacgtaacaagactggaaatttatctctatatgcccatttccggcttccaaggagttctttctggtttcttagttggtgtcaagcaaatcatgcccgaccaggagttgtctattttgaaactgaaagcaaagtggttgccttcccttgcattgttgttctcaattgctataagcttttttacagcagattcagtatcttaccttccaacaatagtatttgggacatatttaggctggatttacctaagatattggcagaagaaaccagagacaaaactaaagggtgacccaagtgatgaattttccttctcttctttcttccctgaatttttgagaccagtaattgatccaattgcaacaatatttgaacggatgctttgtggtagaagatctgagacttctaatgaggaaaggggctatactctaggtggttccacattgcctggttctgatcccattgaagcatcgaggaggagggaaagaggtgcgagagcacttgaagaaagattggcagctgagcttttggcttctggaaagaagggtgaagaatcagaacgaggtgtgaatgataatgtgtag

SEQ ID NO: 29 - DNA, 20 bp, Artificial Sequence
  primer sequence
aagtggttgccttcccttgc

SEQ ID NO: 30 - DNA, 24 bp, Artificial Sequence
  primer sequence
gttttgtctctggtttcttctgcc

SEQ ID NO: 31 - DNA, 41 bp, Artificial Sequence
  primer sequence
ctcggcgcgccgatccaaagaagatccaccattgttcgttt

SEQ ID NO: 32 - DNA, 41 bp, Artificial Sequence
  primer sequence
ctcggcgcgccaaatggtgacagaaactccaagaagcatca

SEQ ID NO: 33 - DNA, 4965 bp, Solanum lycopersicum
ggcgcgccgatccaaagaagatccaccattgttcgtttccaagcttatttattaatttatttattttcctctattttaatttgacggtgaaatatgattatcaattctttcttccattttaataatcaaaattacttaaaatatccaaaatgattgtaattttacaaaaaataaaatgaaattaaataatattaagttttcactttgcctttaatattaagtttttacggaagcaaccattatactaaacaataggaagaaaacaatataaatccgaacaaataaataattatcggttgatatcatgattttcaaatataaaaaagattaattcatattcagtcatatttcttaaaaataaataaggacacaaagaaaaacatgagaattattaataatatagaatgaatgaagcatgttgaatttatatattatataaaatatattttttaattagtctcaattaatatttatattttcaattttaaaaattcataaataaaaaattaaatttacataaaataaaataaattaatacatatatttaacactatataaaacaaatatatgtcacgtaaattttgtttactgttactttattgacgtatattaaatgttagtaataggataaaaactttcaatcattgcaaggaaaagatggtgtatgcattgtatttagcaattaattttatgaataatattaatgcctaatttatgtatttttatatttttttaagtgacaataaattaatgtgacgcaattatgctgtctcaattgtcgagcccctgcaatattactgataacgcaaatgccttgaatgttgattaatctgtaaaaaaagataaagcaagtaccaaagcaaataacagaaattagacatgacaagagacctcacccagatgtaagtttgtactaattttattttttatcttaaattatttatcatatcttgataagttgattagcgtagacacttaaatttgtataaagttgaacaaataggcacacttgaccattttatatggtaatttatatcttacgtgtactatgtcacatatggctcatgtgtctacctgtttaattttatataagtttaagtttttacttgtgtgcattcaaaattgagatgcataattgtgtaactaatttaaattaaatgagacatttaccatgtcttgtgtcatttaaaaataaaaaatacttactattagagaaaatctctctcgttaaaatttgaatggtgacaaataaattgaaacgagaaaaaagaatttgctaaggagaaaagcatggagtttgcaggtagaaggtgacatagaattaggagtactcataatttgcatcattcattgggcagaagttgaataataataataaaagctgatagcagagtcaaatcagattgcagaatcgcactatttttgcgtcattttcgactctatgtgtatgtaacacggtaaactaaaagattcaccaactccacaccccaaaacatatggtgaatcttttgaagactagagtagggatatcgccaaatatgtcctaagaagctgaggactaagtttaatatatccataatttaaatttatagatggagtactagataattttatattaaactaatttttatcaaatccaacctaattcattttttaagtagtattcaaactttgcccatttctaagtcatacttatatttacttttgactttttttccctatagtttggagcttaagttctgagtcagagttaaaacttgaaattaaagttggattgctcactggttaaggttggatctcaggtcgagatcggtttattgttcaggactcgagacaaagtcatctcatgccccaaatccctgtcgggtatcgagtcccaagtaagaattgaatgtcgaatcacgagtcttgaatttcaaattaagcccgaagctcaattatcgagtctaaaatctcgagtcagaggcgaggtcgagtctagagtcctggtcagggttagatttcaagtaaggatcaaaattgagactcaaaatctcaaattttataactccattcaaaatcaaatcaagtctgagatccggactcaaggatcgaattagagaaataaagttgttcattctaaccccctaatgggaaaatgaaggggggggggttggtagggcaataatgaataaatggggtggtgggcatggggccaatgtttgcagggcgtgggttaggactgaacatgacattgtcggttgttttcttatgcacacttcatccagtatctctttgctgtttcccactttctctctcttctctctctctctcccttcttctttagctccgtttgaagcaaccatgttgtctcttgtttcttacatcccatgtttcttcctccccaacaacaccactttttattccttaaaatctctccttcttttttaacaacatagtccattgtttttccttctctacttctagggccccccatcctcctttcatttcttcaatttcacacccccccacataacattagacatagggttagggtttctttttttttttttttaaattaacaaaaaaaaaaaaatcatggcattaggtggtggtaatgaggaaaaagagatgagaatttcaggttttcaaaatatagatcttgtctcacctggtggtgcagcgccagacgacatcgtcagcgctgcaccacacaataattccactactggatctaataaattgaacaagggaactagtacagtgagaggtagatatcgtgagtgcttgaaaaatcatgctgctaatattggtggaaatgttacggatggttgtggtgagtttatgcctagtggtgaagaaggtacactcgaagctttgaaatgtgctgcttgtaattgtcataggaattttcatagaaaagaaatacctaacaacgttggcgctggtgttggtggtggtgttggtggtgataataataataatgcggggattatggttgtgcatccattacagctgccacaaccactaccttctccgataccgtcgttgaatcatcatcaccaccatcaccatcaacacggaggaagatcaatttggactactatgccacctcagcctgtcaaaatggccttcggaggtaatgtgtttagctcatctaaaaattttaatgaaatctcatttgctcaatttgaattaccatgccacctcagcctatcaaaatgggctttggggggtttcgatttaacattcgattatttcgtctaatagtttaaattgtgagacatattttttttatttacttgtactaattatttcattttatgtgtctttgtttcacattgcactaaatttagaaggtgaatatctaatttgaatattgtgattttaaaccttattaatcatttcttaaaatagtatcattaagaataattagcatcatgagaatgttagatttcaaagcttacttaaatactcatatgataagatataaacatacattactagaaataaataaaaaaaattcaccttgaattcgtgagaaatttgtattatcgaactgatttttccactcatttttcacctaaccagctcacggccgcgagaaacacatctaacttcctaattttttcgcgagaaaatgttctggtcattccttactaattcaatcaaaatatctctaaatatttctccatgaaagaaataataattttttagtaatgatatttactgaagacacaaattaaaataaattattacgcttaattatgtcttcaacacgttgttagtacctttttgtcgactcgaacctaagactcttgatatcatgatgaatcgtatgataagttaaaatcattaaagtgaactatatacctacatttttatttttttatttacttaattatatcttcaaacaggtagcggcggaggaagtggggcaacagactcatcaagtgaagaacttaatttcaacacatatcatcatcaacaagcaacatcagtacctccacaacaaccatttatgttggctaagaaaagatttaggacaaaatttagtcaagaacagaaggaaaaaatgttggaatttgctgaaaaattaggttggagaattccaagagaagatgatgctgaagtacaaagattttgctcacaagttggagtcaagagacaggtttttaaggtttggatgcataataataagaacccttcttctgctaaaaaaaatataatacaagaagatcaaccttaattaattttgtttaattaaggttttgagggaaaaaatgaaattaaatttcttgggatttggtttatgcttatgtaatcttactaggaatcgtacgtagctagttttttttgttttttttttgggggggtctatgtagtattattgtaactatatggaaccacttaattagatttttatttttattaattgttgtactttggaacctttattcatctgaaaattttcagctatttcttacacttctcttgtcactgaaatttagaccactcatgatgccgctttatcgaggttagcgaggtagagataaaatttacgtatgtaaactttaaaaaaaatgtgctagatatgttgttgtaatatgagccaagttgtatatcgaattctttagctctcaagatagagtaaagtctgtgtatgaagttagttaattgatgatgttgagggagaaacatgagttatttatttttttgaaatataaaaatgacaagtaaaagcgaaaaataaatgttttttttaggaatggaaggatatttaattaatagagagccgttggatgaaaagaaagaatgaatcttagtcgtctaatttgaattatttgaaagagtagtagttttttttatccttagtcttgtcgaatataaaagcttttcctatttaaatttatttattagtttgtgtattgtctatttcaataaattatgatgcttcttggagtttctgtcaccatttggcgcgcc

SEQ ID NO: 34 - DNA, 1600 bp, Solanum melongena
gaaatagggttagggtttcttaaatcttaattagggttttaatctggtaattagaaaaaaataatggcattaaccggtgaggaaaaggaaatgagaatgtcaggttccttaggatatcattcacttgatcaaaacatagatcacactgtcccaccaaacattcaagatcaatctaccggcggaggtagtggtgcagcgccagttgctggcgttgcaccacataattcctccgccggatctaataaattcaataaggtaacttcatcaagagctaggtaccgtgagtgtctgaaaaatcatgctgctaacattggtggaaatgttaccgatggatgtggtgaattcatgcctagtggagaagaaggaactcttgaggctttgaaatgtgctgcgtgtaattgtcaccgtaattttcacagaaaagaactgcccaacaatggtgataataataataacaacccggggattatggttgtccatccactgcaacttcctcaaccacttcctactccaataccgtcgatgaatcaccatcatcaccatcaccaccaacacagaggtcgatcaatctggactaccatgccacctcagcctgtcaaaatggccttcggaggtaatgtgattatttcatctaaaagtttaaattgttagatgaaatacatttttatttacttccattatttcattttatgtgtctatatttcacatgacgctaaatttagaagcataaaaatgtgtatatatagtgaatattgtgattctagaccttacttatcatttctatgaagtatcattaagaacaattagcatgaaaatcttagatttaaatgcttaaatactcataaactactagaaacaaatttggtcgtgaattagttagaaatttgtgctatgtaacttattttttccactcattttcaaccaaattagttcacggaaatcaaaaaatgcatgatgagaaacaattgtcgattcttaatctttttcatatgtaaaacattactagtttcttcctcaccgattccactaaaatataacatttttagtgtgaaattccatgattttttagtagtgagcttttacctttttttagtgaacaaaacatgtcacctcaaacgtaagacgttagtctgttttgatatgatgattactgtacaataatgtcacctagaattgaaaaaatcattaccatatatacatacatacattttttttcaattatttacttaattatgtcttcaacagccagcgggggaagtggggcaacagactcatcaagtgaagaacttaatttcaacacttatcatcatcaacaggcaacctcagttcctccacaacaaccatttttgttggctaagaaaaggtttaggacaaaattcagtcaagaacaaaaggaaaaaatgttggaatttgcagagaaattaggatggagaattccaagagaagatgatactgaagtacaaagtttctgctcacaagttggagtgaagagacaggtatttaaggtttggatgcataataataagaacccttctgctaaaaagaacataaatcaagaagatcaacaaccttaattaattaaattaaggtcaaatgaaat

SEQ ID NO: 35 - DNA, 879 bp, Solanum melongena; identical to SEQ ID NO: 24

SEQ ID NO: 36 - DNA, 31 bp, Artificial Sequence
  primer sequence
ctcggatccaagctttgaaatgtgctgcgtg

SEQ ID NO: 37 - DNA, 34 bp, Artificial Sequence
  primer sequence
ctcggtaccgagctcagtatcatcttctcttgga

SEQ ID NO: 38 - DNA, 473 bp, Solanum melongena
ctcggatccaagctttgaaatgtgctgcgtgtaattgtcaccgtaattttcacagaaaagaactgcccaacaatggtgataataataataacaacccggggattatggttgtccatccactgcaacttcctcaaccacttcctactccaataccgtcgatgaatcaccatcatcaccatcaccaccaacacagaggtcgatcaatctggactaccatgccacctcagcctgtcaaaatggccttcggagccagcgggggaagtggggcaacagactcatcaagtgaagaacttaatttcaacacttatcatcatcaacaggcaacctcagttcctccacaacaaccatttttgttggctaagaaaaggtttaggacaaaattcagtcaagaacaaaaggaaaaaatgttggaatttgcagagaaattaggatggagaattccaagagaagatgatactgagctcggtaccgag

SEQ ID NO: 39 - DNA, 2561 bp, Solanum melongena
gattctcatttaatgtaccaaattatctttttagtctcatgatttgaaacatgtcacgtgaaaaataataattaaaacttttcaaaaaatattaattatttttctaaacagcgcaaaagaaaaagtagattactcattttgaaatgaagggagtaaaatttttcgttttatctctaatgtgtaaatcattttttatgatttattttttggattgcaaagttcaaaatgttttcttcaaactcctttgcatatatagatttgagactagaaaaaaagatcacaaaatttaataatatttattatattttacacacctctttaatttaaaattataaaattaaaataaaactccatactcaatcagcctaagaatttataaaatgaaatgaagaaaacatatctaagaatttataaaaataaaacgaagaaaacatcttttagactaagcagaaatgcatactaaaataaaccctctcgatctctctggtgtcaatttatgtgacatcttttgaaatataaaagttaaacgagtttttctttactacaacctttacataatcttgtttgtacctttaagaaatttaattttggaatttaaattgcgccacaaaaattaaactcataattataaaaaaattaatatttttgcttttgagattgatatatttgagttgagggtcttccagaaacagtatcttcatctgtataatatagtggtaagatctgtataaattttaccatctctacacttcacttatagaatttcactagatatattattgtcgtttttcttagaatagaaagaaagggagtggttaccattgattgtatgaaagaaaaggttgcttgtgggaaagcatggagtttgcagagttttgcaggtagaaggtgacatatatacaatacaattactcataattttcataattcatttggtagaacgtgataataaaagctgagcagagtcaaatcagattgcaggacagaagaatcgcactatttttgcgtcattttcatctaacacgctaaaataaaagattcaccaaaattaacaagttactgcttattacaaccaagtaacttatatgatggaaaaagtttttacctcagagagtgagtaaaattttccatgaactcaaaatataacaatcaccaaattggaaactgaaaaaagtgatcgtatgatctaaaatttgatggctcattcaattttttcaaaactttatggttttaagttgatttctaaaatgatctttaatttaactcaatttaagttattttcttagatttacttttatctaggctttactacttaagatttactttcattttttatatgggttggggctcaagtctgaagttagatcttgagcatagtcgaagttggatcgctagtcaggattgagtctcaagtcaagtttggttcggtgcttgggacaaagtctaagttagggtctcatgtcctgaactagggtcgagtttcaagtgaagatcaggtcctaggttagagtcgatagttgaaatcttattcttataagatcccaagttagggtcaaggctcaggcctcaatactcaaatctcgagttagggggtgggagaaagggagtttcgaggctgaatctgaagtcagatcaggtctcaagtgattacgaaccaagttttgggttaggtacaagctcgaattccaagtttttaaactcaagatcaagatagagcattgagtcgaagatcaaatcgaggtcgagtgatctcaagaaatactacaagagaattttagatacatatatattaaattttaatttaaaataaggagtgcctaattaggaaaaatataaagtaagtcattctaatttgtacccacctccccaaagagggtggtgggggtggggcaataatgaaggagggtggtgggaatggggccaatgttggcagggcgtgggagaggactgaacatgacattgtcggttgttttcttatgctcagtccttttgtttcttaacaatccataatctcaccacagtttatctttgctgttccgcactactctctccctttcttatctcctctctcttttccttttgaaattatctctctttttcttttagctccgtttcaagcaaccatgttgtctcttgtttcttacattctatgttgcctcctccccaacacaccacaaccttaaaacaaacctcatcactttaaatttctctttcctccatctctaactcactactgtttcttaatctcaatttcatgcatttttaaggtggtggtccgccggcttttgcttcttctaaggtagtagttctagggccccatcttctttatttccagcttaatttcttcttctgttttaccctttttccttttctctttctctctcaccccacataccattaagtagaatcagaaggtttcatcatttttgttagttacatctatttttcttcagtgttttgttagaataagaaatagggttagggtttcttaaatcttaattagggttttaatctggtaattagaaaaaaataatggcattaaccggtgaggaaaaggaaat

SEQ ID NO: 40 - DNA, 29 bp, Solanum melongena
atggcattaaccggtgaggaaaaggaaat

SEQ ID NO: 41 - DNA, 1153 bp, Solanum melongena
gagaggcaaccccatctctcactcgtctgaaacacgcatcaccattaaagctctctgctttctgtgatcacttggcactatgagtatgagtatgtatacggggttcaccaggctttgcaagggtcttgcggttgtgcttgtgggctgtcacattgttgttcagattcttccttctgctctttcgtatcttgctcttatccctgccaagactattccatttgcatggaatcttataacagctggttacactgaacaaacagtgtatggggcaatcatcagcactatcggacttctttacctggggaagctgcttgagcccatttggggttctagggaattcttgaagttcatctttattgtcaactttttgacatctgtctgtgtttttatcacagccatatcattgtactacataacaagactggaaatttatctctatatgcccatttccggcttccaaggagttctttctggtttcttagttggcgtcaagcaaatcatgcctgaccaggagttgtctattttgaaactgaaagcaaagtggttgccttcctttgcattgttgttctcaattgctataagcttttttacagcagatttagtatcatatcttccaacagtagtatttgggacatatttaggctggatttacctgagatactggcagaagaaaccagagacaaaattaaagggtgacccaagtgatgaattttccttctcttctttcttccctgaatttttgagaccagtaattgatccaattgcaacaatatttgagcggatgctttgtggtagaagatctgagacttctaatgaggaaaggggatatactctaggtggttccacattgcctggttctgatcccattgaagcatcgaggaggagggaaagaggtgccagagcacttgaagaaagattggcagctgagcgtttggctgctggaaagaaggctgaagaatcagaaagagatgtgaatgataatgtgtaaaagctggtcaatatatatgtaatgcgctttctttgcagaaagaacgtttgtccgctagaagctagaaattgtgcactttttggcagtgtactcttttggattcttgacggaagttgtcgtctgttgtttatcatccctatggtaaatgtacatttttctccttcc

SEQ ID NO: 42 - DNA, 729 bp, Solanum lycopersicum; identical to SEQ ID NO: 28

SEQ ID NO: 43 - DNA, 20 bp, Artificial Sequence
  primer sequence
aaagaactgcccaacaatgg

SEQ ID NO: 44 - DNA, 20 bp, Artificial Sequence
  primer sequence
gatgagtctgttgccccact

SEQ ID NO: 45 - DNA, 20 bp, Artificial Sequence
  primer sequence
aagtggttgccttcccttgc

SEQ ID NO: 46 - DNA, 24 bp, Artificial Sequence
  primer sequence
gttttgtctctggtttcttctgcc

SEQ ID NO: 47 - DNA, 1039 bp, Solanum lycopersicum
tataataaaatatcttatgaaaaaaatatcacaaaaaatcagttttcaaaaatatttacaaaaataatttaaagttagattaaaatttaaataatctcgctaaaaataatttacgtgtaaatcaaactctaaaatcatcctcaacgctcaaagtggtcatattggacagtggatggagtgggaaataatatttctgacatataattattctaaaaaaataatattatgtgtgtaaattgtaaattaactaaaatttttatgatatattttaaatttataatttcattaaaagaacttattttattaaaaatatatctatagtaaataaacttaattaaaatttcactcatcattaattaaaattttaaattcgcatctcagacaaaataaaatatactccacaaactttgttacactttcgacaagaatatttatgggaatttctcgtctcctttttcaattaggatttagttgagttgggtagaatcaaataaaaaaattatatatatatatatatatatatatatatatatatatattcctaaatgagaagaagatgtagaaattcccaaattaatctatatcactttctatatgtttatcctttaatcattttcaattttacgtagtaatatcgaccattacataagttattaatggctgagagaaagcgagtagatgatcataatacagtatataagacttgtgattcagattattcaatcttccatgcaaattgtccaatatattgttcacttgtggttccaattattccacttcctctattaaattagctttgattatagcaagcagtggtggagtcaggatttttaataagggagttcaaaatatgaaaaataaatatatgaattagtggaagggggtttaaacatttatcatacatatctaaaaaataattttaatcatatataaatttcctaagtatatgttacctccgctcctgataatagatattctttaaaattaatcgagatgccaatatattaatcgagatatcatgctatatttattggaaaaa

SEQ ID NO: 48 - DNA, 21 bp, Solanum lycopersicum
ggaatttctcgtctccttttt

SEQ ID NO: 49 - DNA, 21 bp, Solanum lycopersicum
tactcgctttctctcagccat

SEQ ID NO: 50 - DNA, 249 bp, Solanum lycopersicum
tttgacatgccaacttctttttgtaaagtggatatatgattaactttaaaaattaaaatattttttagagaaaaatcgagaaaaaaaattttgcacaaattttactacacaaaataggcagaattattttttgagcgatttaaaaagaaaaaagattaaaaaactaatttgaagcagtcgtcgctaagatgtcaagtgtctggttgtgcattatgaaagttggagtctacttaaagtgtctggttgtgc

SEQ ID NO: 51 - DNA, 379 bp, Solanum lycopersicum
ttttgatatattggtgcccctgtcgttcaaaaactagaacatatatgcacttcactctaacgaaagattaaataaaaatatgtggcataattttattcatcgatccgacatcgaattggtaggtaaaattgtgatatatgtatgtttgctaatataaaggatatgtatattttaatttttgaacggcaaaaaacaatcgaagggttacaatatgttatattaaagcctatcgtatatattaagatccactttccagcatatattccctattttatgtttcttctctgaaacttcaagaaggatctatgtagaaatatactatagtgttataagtaatacttattaattaattactccataggaaattggggaggaggta

SEQ ID NO: 52 - DNA, 360 bp, Solanum lycopersicum
tggatggctaccatgttcatacagaaagatcaacttttacaaatttaatatatacatacatatatatatatatatatatatatatatataaatatatatatatatatatatatatatatatatatatatataaattacaaatttcttttattatttcacctgatgtatatattgaatttaaatttaaatttaaatacaatatcatacttaatacttacattcaaaatatctaattactgataaataaatatttattgctatatcataattctgattgctgtttatatatcgtatatatgagtactttaaaatatctatcttaccaaatatcattcaaaaacaatgaagtggggagaagaa

SEQ ID NO: 53 - DNA, 22 bp, Solanum lycopersicum
aggatggctaccatgttcatac

SEQ ID NO: 54 - DNA, 24 bp, Solanum lycopersicum
gtttcttctccccacttcattgtt

SEQ ID NO: 55 - DNA, 1041 bp, Solanum lycopersicum
aaatttcaagttgggatattctaaaatgttatatgaatctttttattataatatcatatgttatcgttatacttgagtacgattatatttttcttttattaaatatgagattctgagagagacacataccacatgatttatgaggaaaaagtaatgttacatcaaggtccaaaaacttaggcaaggaccatatatgattgtaggtaagaatatttttatttacataatacataaaagaacaagttaattttggactttctttttttataagaaaaatactagaagagggattttcaaagtccactatgaaaagtagaaatcaaagtgtaaaaaaattattggaaagttgaggatagtggggctaagtctataaatagtttctcccaaagttgttctcttgcatatggccaacatgtatatacaaatttaggtggaataatgtatattaatttgggcccttgtattctctcctacttgctatttcactctcctcaactttcatatatatatatatatatatatatatatatatatatatattaaaattttatgagttcagataaaccgttgcatcgaaaaagagttacgagataacacaacatgagatagtgttccatctttcaacttttttaacatatgcataatgttgagtgcaccaaaatcagaaacaagataagggtacaagaaaaaacagttatccattttgctaacttagttggtatactgattgaagcccaacattaaaaaagtgttctttttgttcaatcatgttcatattttgggataaccttagcttcatagtggaatgacacctatgtactatgtttggctttggtctctttcacatcagatggaggaattggacaactaaaatcttgataacttccatgaaaactaaaatcatgtttccatatgatgttttggatggagaatgtgtttgcataagacaacaagacaagatttaacagattatatagcaaaaagcaaagtggcgtccgagaccggttcttcgttctttcaaatcgaaagaaagctagagga

SEQ ID NO: 56 - DNA, 22 bp, Solanum lycopersicum
atgaggatagtggggctaagtc

SEQ ID NO: 57 - DNA, 25 bp, Solanum lycopersicum
gtttcgtaactctttttcgatgcaa